(19) United States
(12) Patent Application Publication
(10) Pub. No.: US 2025/0098992 A1
(43) Pub. Date: Mar. 27, 2025

(54) WEARABLE DEVICE AND ELECTRONIC DEVICE FOR ESTIMATING PERCENTAGE OF TARGET MATERIAL, AND METHOD FOR OPERATING SAME

(71) Applicant: SAMSUNG ELECTRONICS CO., LTD., Suwon-si (KR)

(72) Inventor: Hyunjun JUNG, Suwon-si (KR)

(21) Appl. No.: 18/973,120

(22) Filed: Dec. 9, 2024

Related U.S. Application Data
(63) Continuation of application No. PCT/KR2023/004360, filed on Mar. 31, 2023.

(30) Foreign Application Priority Data
Jun. 13, 2022 (KR) 10-2022-0071493
Sep. 6, 2022 (KR) 10-2022-0112944

(51) Int. Cl. A61B 5/1455; A61B 5/00

(52) U.S. Cl. CPC A61B 5/1455; A61B 5/681; A61B 5/7225; A61B 5/7275; A61B 5/74

(57) ABSTRACT

A wearable device includes a light-emitting part including a light source for emitting light of multiple wavelengths including a first wavelength band corresponding to a first bandwidth, a second wavelength band corresponding to a second bandwidth, and a third wavelength band corresponding to a third bandwidth, a light-receiving part for detecting reflection light reflected from the user's skin, by light of multiple wavelengths, a signal acquisition part for selecting light of two or more wavelengths at which an oxygen hemoglobin absorption rate is the same as a reduced hemoglobin absorption rate, from among the reflection light, the signal acquisition part acquiring pulse wave signals corresponding to the light of the selected wavelengths, and an estimating part for estimating a percentage of an object to be detected, on the basis of an absorption rate of each wavelength of pulse wave signals.

### CROSS-REFERENCE TO RELATED APPLICATIONS

This application is a continuation application, under 35 U.S.C. § 111(a), of International Patent Application No. PCT/KR2023/004360, filed on Mar. 31, 2023, which claims priority to Korean Patent Application No. 10-2022-0071493, filed on Jun. 13, 2022, and Korean Patent Application No. 10-2022-0112944, filed on Sep. 6, 2022, the content of which in their entirety is herein incorporated by reference.

### TECHNICAL FIELD

The disclosure relates to a wearable device, an electronic device, and an operating method for estimating a percentage of a target material.

### DESCRIPTION OF THE BACKGROUND ART

Carbon monoxide (CO) is colorless and odorless, which makes it difficult to notice its presence by sense alone. Carbon monoxide poisoning may cause fatal consequences that are directly contributed to deaths. Carbon monoxide (CO) has a binding strength that is 100 times stronger or more than that of oxygen (O2) and hemoglobin (Hb), which interferes with the binding between oxygen (O2) and hemoglobin (Hb) and reduces the delivery of oxygen to human tissues. Carbon monoxide (CO) may generate carboxyhemoglobin (COHb) when inhaled into a human body and bound to hemoglobin. The detection of carboxyhemoglobin (COHb) may be used as an important indicator to determine whether carbon monoxide poisoning occurs.

### SUMMARY

When detecting carboxyhemoglobin (COHb) in a non-invasive method without penetrating the skin or passing through any orifice of a body, a finger-type detection device has been used. However, during daily life when fingers are used a lot, a measured part has relatively low contact stability, and measurement without awareness gets difficult. Thus, the detection device is not readily available during daily life. In addition, a method of using a multi-spectrum for the detection of carboxyhemoglobin may show the composition of different spectra, but the complexity of a system is high, which makes the use of the system practically impossible.

In embodiments, a wearable device may detect the concentration of carbon monoxide in the air, based on a biosignal (e.g., a photoplethysmography (PPG) signal).

In embodiments, the wearable device may detect whether carbon monoxide poisoning occurs by estimating a percentage of carboxyhemoglobin (COHb).

In embodiments, the exposure risk to carbon monoxide during a user's daily life, including sleep, may be monitored continuously. The risk may be notified to the user, an external agency (e.g., an emergency medical agency), and/or an external electronic device.

However, the goals to be achieved are not limited to those described above and may be expanded in various manners within the scope without departing from the spirit and field of the disclosure.

In an embodiment, a wearable device includes a light emitter including a light source that emits lights of multiple wavelengths including a first wavelength band corresponding to a first bandwidth (420 nanometers (nm) to 460 nm), a second wavelength band corresponding to a second bandwidth (500 nm to 540 nm), and a third wavelength band corresponding to a third bandwidth (785 nm to 825 nm), a light receiver configured to detect at least some of reflected lights that are reflected from a user's skin by the lights of multiple wavelengths, a signal acquirer configured to select lights of two or more wavelengths, in which an absorption rate of oxyhemoglobin is the same as an absorption rate of a reduced hemoglobin, from among the reflected lights and acquire pulse wave signals corresponding to the selected lights of wavelengths, and an estimator configured to estimate a percentage (%) of a target to be detected, based on a ratio of an absorption rate of each wavelength of the pulse wave signals.

In an embodiment, an electronic device includes a communication interface configured to receive pulse wave signals corresponding to lights of two or more wavelengths selected from among reflected lights by lights of multiple wavelengths from a wearable device, a processor configured to estimate a percentage of a target to be detected, based on a ratio of an absorption rate of each wavelength of the pulse wave signals, and calculate risk information corresponding to the target, based on the percentage of the target, and an output device configured to provide a notification to a user according to the risk information.

In an embodiment, an operating method of a wearable device includes irradiating a user's skin with lights of multiple wavelengths from a light source, detecting reflected lights reflected from the skin by the lights of multiple wavelengths, selecting lights of two or more wavelengths, in which an absorption rate of oxyhemoglobin is the same as an absorption rate of a reduced hemoglobin, from among the reflected lights, acquiring pulse wave signals corresponding to the selected lights of wavelengths, estimating a percentage of a target to be detected, based on a ratio of an absorption rate of each wavelength of the pulse wave signals, and notifying risk information corresponding to the target, based on the percentage of the target.

In an embodiment, by mounting a plurality of light-emitting diodes (LEDs) having different wavelengths, the detection of carbon monoxide may be performed by a commercialized wearable device without a separate additional device.

In an embodiment, by monitoring the detection of carboxyhemoglobin (COHb) during daily life temporarily and/or continuously, a wearable device may perform health management (e.g., smoking management or smoking cessation assistance for smokers), besides the detection of the poisoning of a target material, such as carbon monoxide.

The effects to be achieved are not limited to those described above, and other effects not mentioned above will be clearly understood by one of ordinary skill in the art from the following description.

### BRIEF DESCRIPTION OF DRAWINGS

The above and other embodiments, advantages and features of this disclosure will become more apparent by describing in further detail embodiments thereof with reference to the accompanying drawings, in which:

FIG. 1A is a front perspective view of an embodiment of a wearable device, and

FIG. 1B is a rear perspective view of an embodiment of the wearable device.

FIG. 2 is an exploded perspective view of an embodiment of the wearable device.

FIG. 3 is a block diagram illustrating an embodiment of the wearable device.

FIG. 4 is a diagram illustrating the structures of a light emitter and a light receiver.

FIG. 5 is a diagram illustrating an embodiment of an arrangement relationship between a light emitter and a light receiver of the wearable device.

FIG. 6 is a diagram illustrating absorption spectra of hemoglobin in various states by a plurality of lights of wavelengths.

FIG. 7 is a diagram illustrating an embodiment of the intensity of the light absorbed into and reflected from a human tissue.

FIG. 8 is a diagram illustrating an embodiment of the principle of detecting blood carboxyhemoglobin (COHb).

FIG. 9 is a diagram illustrating an embodiment of the principle of estimating a percentage of a target to be detected by an estimator.

FIG. 10 is a flowchart illustrating an embodiment of an operating method of the wearable device.

FIG. 11 is a flowchart illustrating an embodiment of the operating method of the wearable device.

FIG. 12 is a block diagram of an embodiment of an electronic device.

FIG. 13 is a block diagram illustrating an embodiment of an electronic device in a network environment.

### DETAILED DESCRIPTION

Hereinafter, embodiments are described in detail with reference to the accompanying drawings. When describing the embodiments with reference to the accompanying drawings, like reference numerals refer to like elements and a repeated description related thereto are omitted.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). The term such as “about” can mean within one or more standard deviations, or within +30%, 20%, 10%, 5% of the stated value, for example.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

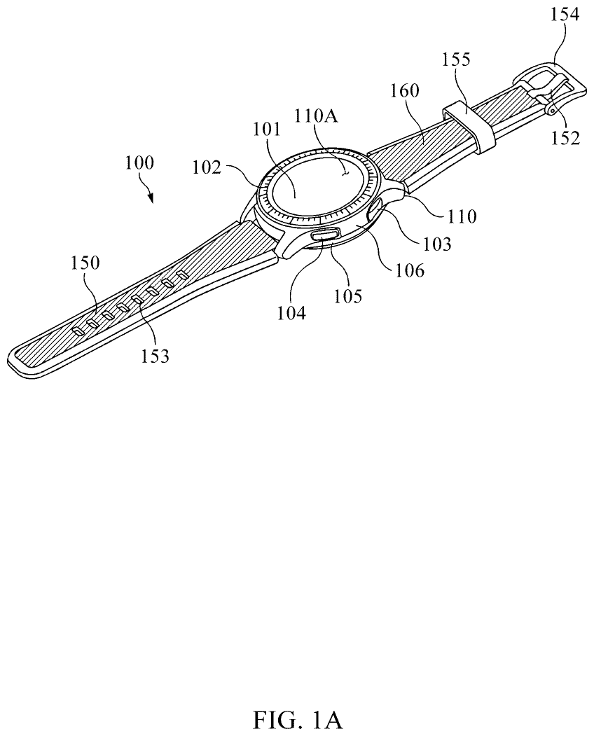

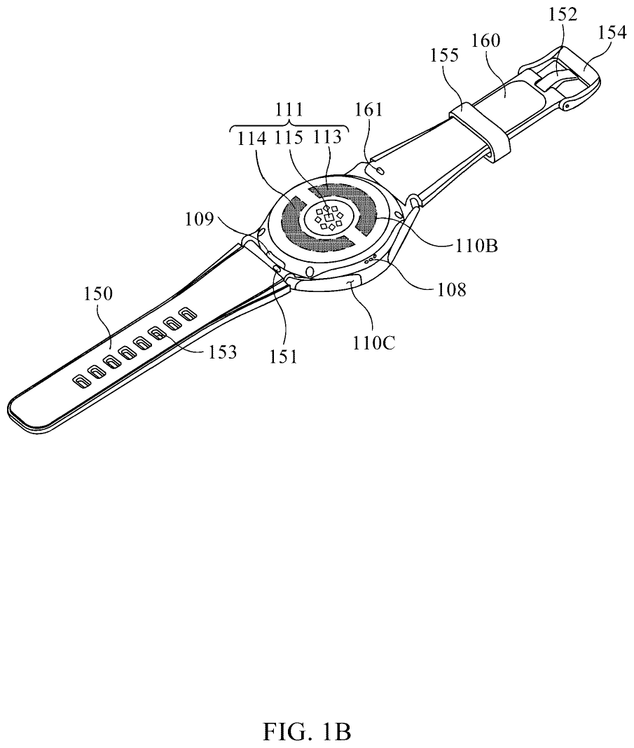

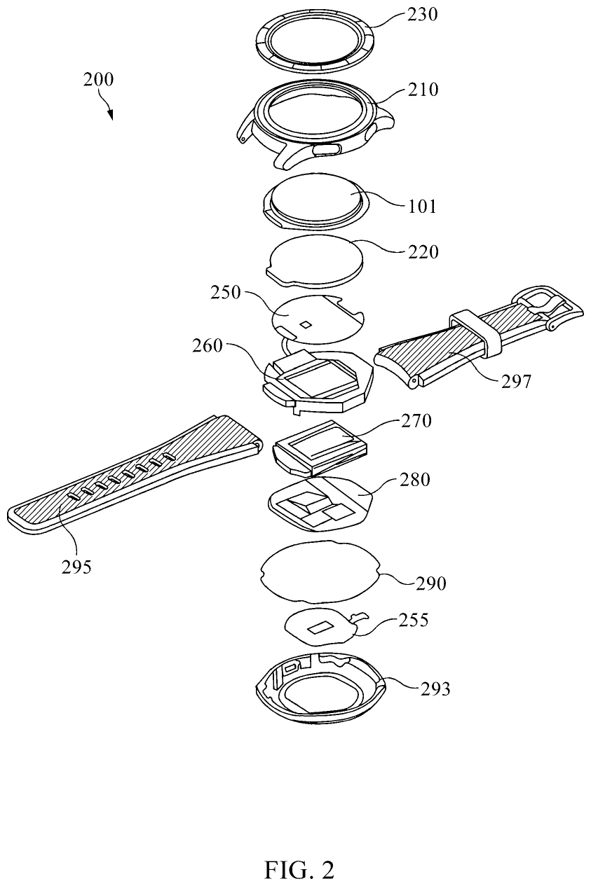

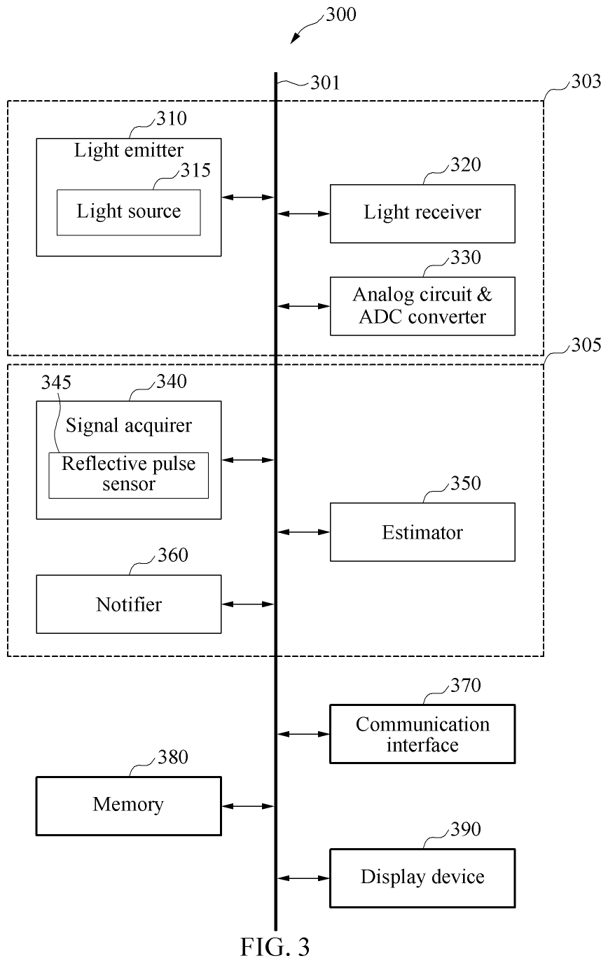

FIG. 1A is a front perspective view of an embodiment of a wearable device, and FIG. 1B is a rear perspective view of an embodiment of the wearable device. Referring to FIGS. 1A and 1i, in an embodiment, a wearable device 100 (e.g., a wearable device 200 of FIG. 2 and/or a wearable device 300 of FIG. 3) may include a housing 110 including a first surface (or a front surface) 110A, a second surface (or a rear surface) 110B, and a side surface 110C surrounding a space between the first surface 110A and the second surface 110B, and fastening members 150 and 160 (e.g., fastening members 295 and 297 of FIG. 2) connected to at least a portion of the housing 110 and configured to detachably attach the wearable device 100 to a body part (e.g., a wrist or an ankle) of a user. In an embodiment (not shown), the housing may also refer to a structure that forms a portion of the first surface 110A, the second surface 110B, and the side surface 110C of FIG. 1A. In an embodiment, the first surface 110a may be formed by a front plate 101 (e.g., a glass plate or a polymer plate including various coating layers) of which at least a portion is substantially transparent. The second surface 110B may be formed by a rear plate (e.g., a rear plate 293 of FIG. 2) that is substantially opaque. The rear plate 293 may include or consist of coated or colored glass, ceramic, polymer, metal (e.g., aluminum, stainless steel, or magnesium), or a combination of at least two thereof, for example. The side surface 110C may be coupled to the front plate 101 and the rear plate 293 and may be formed by a side bezel structure (or a “side member”) 106 including a metal and/or a polymer. In an embodiment, the rear plate 293 and the side bezel structure 106 may be unitary and may include the same material (e.g., a metal material, such as aluminum). The fastening members 150 and 160 may include or consist of various materials and may have various shapes. In an embodiment, woven fabric, leather, rubber, urethane, metal, ceramic, or a combination of at least two of the aforementioned materials may be formed in an integrated form or with a plurality of unit links that are movable relative to each other, for example.

In an embodiment, the wearable device 100 may include at least one of a display (e.g., a display 220 of FIG. 2), audio modules 105 and 108, a sensor module 111, and key input devices 102, 103, and 104, and may define a connector hole 109. In an embodiment, the wearable device 100 may not include or define at least one (e.g., the key input devices 102, 103, and 104, the connector hole 109, or the sensor module 111) of the components, or additionally include other components.

The display 220 may be exposed through some portions of the front plate 101, for example. The display 220 may have a shape corresponding to the shape of the front plate 101 or may have various shapes, such as a circle, an oval, or a polygon. The display 220 may be coupled to or disposed next (adjacent) to a touch sensing circuit, a pressure sensor capable of measuring an intensity (or pressure) of a touch, and/or a fingerprint sensor.

The audio modules 105 and 108 may define a microphone hole 105 and a speaker hole 108. A microphone for acquiring an external sound may be disposed in the microphone hole 105. In some embodiments, a plurality of microphones may be disposed to detect a direction of a sound. The speaker hole 108 may be used as an external speaker and a call receiver for calls. In some embodiments, the speaker hole 108 and the microphone hole 105 may be implemented as a single hole, or a speaker (e.g., a piezo speaker) may be included without the speaker hole 108.

The sensor module 111 may generate an electrical signal or a data value corresponding to an internal operating state of the wearable device 100 or an external environmental state. The sensor module 111 may include a biometric sensor module 111 (e.g., a heart rate monitor (HRM) sensor) disposed on the second surface 110B of the housing 110, for example. The wearable device 100 may further include at least one of sensor modules (not shown), e.g., a gesture sensor, a gyro sensor, an atmospheric pressure sensor, a magnetic sensor, an acceleration sensor, a grip sensor, a color sensor, an infrared (IR) sensor, a biometric sensor, a temperature sensor, a humidity sensor, or an illuminance sensor.

The sensor module 111 may include electrode areas 113 and 114 that form a portion of the surface of the wearable device 100 and a biosignal detection circuit 115 electrically connected to the electrode areas 113 and 114. In an embodiment, the electrode areas 113 and 114 may include a first electrode area 113 and a second electrode area 114 disposed on the second surface 110B of the housing 110, for example. The sensor module 111 may be configured such that the electrode areas 113 and 114 obtain an electrical signal from a body part of the user, and the biosignal detection circuit 115 detects biometric information of the user based on the electrical signal.

The key input devices 102, 103, and 104 may include a wheel key 102 disposed on the first surface 110A of the housing 110 and rotatable in at least one direction, and/or side key buttons 103 and 104 disposed on the side surface 110C of the housing 110. The wheel key 102 may have a shape corresponding to the shape of the front plate 101. In an embodiment, the wearable device 100 may not include some or all of the above-described key input devices 102, 103, and 104, and the key input devices 102, 103, and 104 that are not included may be implemented in other forms, such as soft keys on the display 220. The connector hole 109 may include another connector hole (not shown) that accommodates a connector (e.g., a universal serial bus (USB) connector) for transmitting and receiving power and/or data to and from an external electronic device and accommodates a connector for transmitting and receiving an audio signal to and from an external electronic device. The wearable device 100 may further include a connector cover (not shown) that covers at least a portion of the connector hole 109 and blocks the infiltration of external foreign materials into the connector hole 109, for example.

The fastening members 150 and 160 may be detachably fastened to at least a partial area of the housing 110 using locking members 151 and 161. The fastening members 150 and 160 may include or define one or more of a fixing member 152, a fixing member fastening hole 153, a band guide member 154, and a band fixing ring 155.

The fixing member 152 may fix the housing 110 and the fastening members 150 and 160 to a part (e.g., a wrist or an ankle) of the user's body. The fixing member fastening hole 153 may correspond to the fixing member 152 to fix the housing 110 and the fastening members 150 and 160 to the body part of the user. The band guide member 154 may limit a range of a movement of the fixing member 152 when the fixing member 152 is fastened to the fixing member fastening hole 153, so that the fastening members 150 and 160 may be fastened to the body part of the user in a state of being brought into close contact with the body part of the user. The band fixing ring 155 may limit the range of movement of the fastening members 150 and 160 in a state in which the fixing member 152 and the fixing member fastening hole 153 are fastened with each other.

FIG. 2 is an exploded perspective view of an embodiment of the wearable device. Referring to FIG. 2, the wearable device 200 (e.g., the wearable device 100 of FIG. 1 or the wearable device 300 of FIG. 3) may include a side bezel structure 210, a wheel key 220, the front plate 101, the display 220, a bezel 230, a first antenna 250, a second antenna 255, a support member 260 (e.g., a bracket), a battery 270, a printed circuit board (PCB) 280, a sealing member 290, a rear plate 293, and the fastening members 295 and 297. At least one of the components of the wearable device 200 may be the same as or similar to at least one of the components of the wearable device 100 of FIG. 1 and/or the wearable device 300 of FIG. 3, and a repeated description thereof will be omitted hereinafter.

The support member 260 may be disposed inside the wearable device 200 and connected to the side bezel structure 210 or may be formed integrally with the side bezel structure 210. The support member 260 may include or consist of a metal material and/or a non-metal material (e.g., a polymer), for example. The display 220 may be connected to one surface of the support member 260, and the PCB 280 may be connected to an opposite surface thereof.

The PCB 280 may be provided with a processor, a memory, and/or an interface disposed (e.g., mounted) thereon. The processor may include, e.g., one or more of a central processing unit, an application processor, a graphics processing unit (GPU), a sensor processor, or a communication processor. The memory may include a volatile memory or a non-volatile memory, for example. The interface may include a high-definition multimedia interface (HDMI), a USB interface, a secure digital (SD) card interface, and/or an audio interface, for example. In an embodiment, the interface may electrically or physically connect the wearable device 200 to an external electronic device and may include a USB connector, an SD card/multimedia card (MMC) connector, or an audio connector, for example.

The battery 270, which is a device for supplying power to at least one component of the wearable device 200, may include a primary cell that is not rechargeable, a secondary cell that is rechargeable, or a fuel cell, for example. In an embodiment, at least a portion of the battery 270 may be disposed on substantially the same plane as the PCB 280, for example. The battery 270 may be disposed integrally inside the wearable device 200 or may be disposed detachably from the wearable device 200.

The first antenna 250 may be disposed between the display 220 and the support member 260. The first antenna 250 may include a near-field communication (NFC) antenna, a wireless charging antenna, and/or a magnetic secure transmission (MST) antenna, for example. In an embodiment, the first antenna 250 may perform short-range communication with an external device, may wirelessly transmit and receive power used for charging, or may transmit a magnetism-based signal including a short-range communication signal or payment data, for example. In an embodiment, an antenna structure may be formed by a portion of the side bezel structure 210 and/or the support member 260, or any combinations thereof.

The second antenna 255 may be disposed between the PCB 280 and the rear plate 293. The second antenna 255 may include an NFC antenna, a wireless charging antenna, and/or an MST antenna, for example. In an embodiment, the second antenna 255 may perform short-range communication with an external device, may wirelessly transmit and receive power used for charging, or may transmit a magnetism-based signal including a short-range communication signal or payment data, for example. In an embodiment, an antenna structure may be formed by a portion of the side bezel structure 210 and/or the rear plate 293, or any combinations thereof.

The sealing member 290 may be disposed between the side bezel structure 210 and the rear plate 293. The sealing member 290 may prevent moisture and foreign materials from being introduced into a space surrounded by the side bezel structure 210 and the rear plate 293 from the outside.

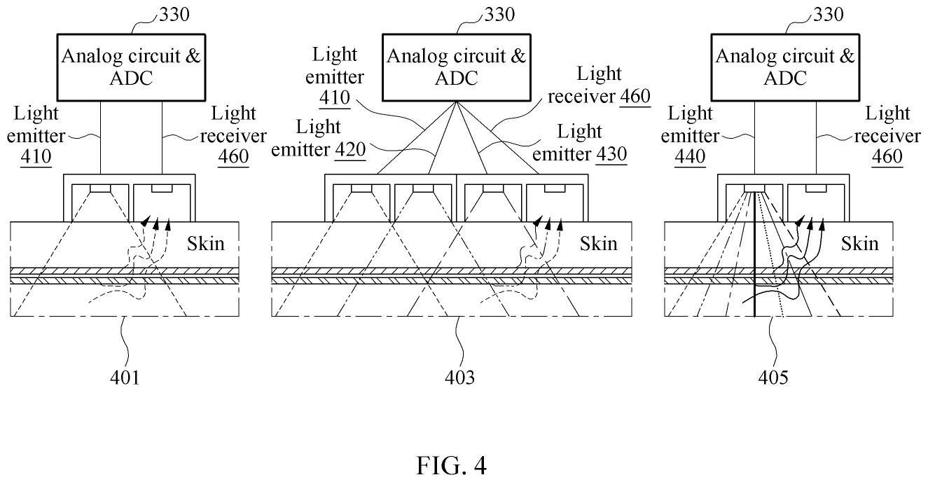

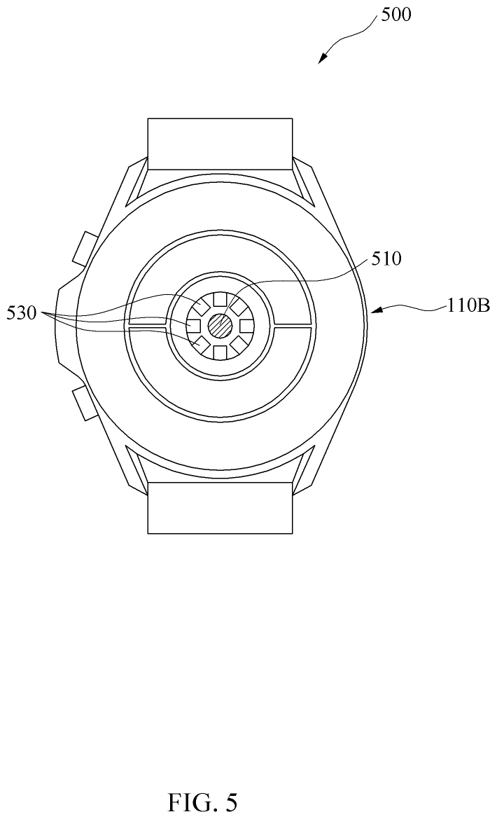

FIG. 3 is a block diagram illustrating an embodiment of the wearable device. Referring to FIG. 3, the wearable device 300 (e.g., the wearable device 100 of FIG. 1 and/or the wearable device 200 of FIG. 2) may include a light emitter 310 (e.g., light emitters 410, 420, 430, and 440 of FIG. 4 and/or a light emitter 510 of FIG. 5), a light receiver 320 (e.g., a light receiver 460 of FIG. 4 and/or a plurality of light receivers 530 of FIG. 5), an analog circuit & analog-to-digital converter (ADC) 330, a signal acquirer 340, an estimator 350, and a notifier 360. The wearable device 300 may further include a communication interface 370, a memory 380, and a display device 390 (e.g., the display 220 of FIG. 2). The light emitter 310, the light receiver 320, the analog circuit & ADC 330, the signal acquirer 340, the estimator 350, the notifier 360, the communication interface 370, the memory 380, and the display device 390 may be connected electrically and/or operationally to one another through a communication bus 301.

In an embodiment, the light emitter 310, the light receiver 320, and the analog circuit & ADC 330 may be formed as a photoplethysmography (PPG) acquisition module 303. In addition, the signal acquirer 340, the estimator 350, and the notifier 360 may be formed as a processor 305.

The light emitter 310 may include a light source 315 that emits lights of multiple wavelengths including a first wavelength band corresponding to a first bandwidth (equal to or greater than 420 nanometers (nm) and equal to or less than 460 nm), a second wavelength band corresponding to a second bandwidth (equal to or greater than 500 nm and equal to or less than 540 nm), and a third wavelength band corresponding to a third bandwidth (equal to or greater than 785 nm and equal to or less than 825 nm). The light source 315 may be a light-emitting diode (LED) and/or a laser, for example, but may not be limited thereto.

The light source 315 may be any one of as illustrated in FIG. 4, an LED light source (e.g., the light emitter 410 of FIG. 4) that generates a light of a single wavelength band, in which a center frequency is one, multiple LED light sources (e.g., the light emitters 410, 420, and 430 of FIG. 4) that generate a light of a single wavelength band, and a multispectral light source (e.g., the light emitter 440 of FIG. 4) that generates lights of multiple wavelength bands in a spectral sensor, for example, but may not be limited thereto.

A fixed light emitter, such as an LED or a laser, or a modulated light emitter, such as a spectral sensor, which may enable light modulation may be used as the light source 315.

In an embodiment, when using the fixed light emitter as the light source 315, a device that generates a wavelength substantially similar to a designated wavelength may be used, for example. In this case, the process of finding a wavelength for detecting carboxyhemoglobin (COHb) may be omitted.

When using the modulated light emitter as the light source 315, the spectral sensor may be a semiconductor-based sensor that performs light modulation into multiple spectra by a modulator. In this case, the light modulation method may include an electro-absorption modulator (EAM) method and/or an electro-optic modulator (EOM) method, for example. The EAM method may perform light modulation by controlling an absorption rate and may be readily miniaturized and used with relatively low power. The EOM method may perform light modulation by controlling optical refraction and may have advantageous effects in implementing relatively high performance and relatively high frequency.

As described above, the process of determining a wavelength for detecting carboxyhemoglobin (COHb) may vary depending on whether to use, as the light source 315, the fixed light emitter or a sensor, such as a spectral sensor, that may perform light modulation. The light emitter 310 may be one light emitter or a plurality of light emitters. In an embodiment, when the light emitter 310 is the plurality of light emitters, for example, the wearable device 300 may operate the light emitters corresponding to different wavelengths at set time intervals and, based on the operation of the light emitters, may acquire pulse wave signals by reflected lights detected by the light receiver 320.

The light receiver 320 may detect at least some of the reflected lights that are reflected from a user's skin by the lights of multiple wavelengths emitted by the light source 315. In an embodiment, the light receiver 320 may detect at least some of the reflected lights (e.g., PPG signals) that are reflected from the user's skin during a time interval including a time when a light of the light emitter 310 is turned on, for example. The reflected lights (e.g., the PPG signals) that are reflected from the user's skin may also be also referred to as ‘backscattered lights’. In this case, an increase or decrease of blood in a vessel on a light path may cause an increase or decrease in the number of the reflected lights.

The light receiver 320 may detect reflected lights by a light-sensitive element, such as a photo diode and/or a photo transistor, for example, but examples are not limited thereto. The light receiver 320 may receive at least some of reflected lights corresponding to multiple wavelengths and may output a resistance value changed according to the intensity of the received reflected lights.

In embodiments, the structure of the light emitter 310 and the light receiver 320 is described in detail below with reference to FIG. 4, and the arrangement of the light emitter 310 and the light receiver 320 is described in detail below with reference to FIG. 5.

In an embodiment, the wearable device 300 may further include a circuit (not shown) that adjusts a driving signal of the light emitter 310 such that the light receiver 320 may receive an electrical signal of sufficient intensity after receiving feedback on a reception value of the light receiver 320. Since the reflectivity of a light varies depending on persons, the wearable device 300 may adjust the amount of light applied to a human body through the circuit.

The analog circuit & ADC 330 may perform signal processing including amplification and filtering for reflected lights detected in the light receiver 320 and may convert a signal-processed reflected light signal (analog signal) into a digital signal. The analog circuit & ADC 330 may include an amplification circuit that amplifies a signal of reflected lights and/or a filter that filters a signal of a predetermined frequency band of a reflected light signal. In an embodiment, the analog circuit & ADC 330 may remove noise that is greater than or equal to 50 hertz (Hz) by a low-pass filter of 50 Hz, for example. In addition, an analog-to-digital (ADC) converter may convert amplified and/or filtered reflected lights into a digital signal.

The wearable device 300 may enable the light emitter 310 to emit lights of different wavelengths at set time intervals and may acquire PPG signals of multiple wavelengths by acquired signals (e.g., reflected lights) from the light receiver 320 when the lights of different wavelengths are emitted.

The signal acquirer 340 may select lights of two or more wavelengths, in which an absorption rate of oxyhemoglobin is the same as an absorption rate of a reduced hemoglobin, from among the reflected lights. The ‘absorption rate’ refers to a ‘light absorption rate’ herein. Even without separate description, the absorption rate may be understood as the light absorption rate.

The signal acquirer 340 may acquire a pulse wave signal corresponding to the selected lights of wavelengths. ‘Oxyhemoglobin’ may be hemoglobin bound to oxygen, and ‘reduced hemoglobin’ may be hemoglobin dissociated from oxygen. Here, the reflected lights may be reflected lights in an analog signal form detected in the light receiver 320 or reflected lights converted by the analog circuit & ADC 330 into digital signals, for example.

The signal acquirer 340 may select lights of two or more wavelengths, in which a first absorption rate of a light corresponding to a target to be detected is not changed by a second absorption rate of oxyhemoglobin and reduced hemoglobin, from among the reflected lights.

The ‘target’ to be detected may be any one of carboxyhemoglobin (COHb) and methemoglobin (MetHb), but examples are not limited thereto. Methemoglobin (MetHb) is a variant of hemoglobin (Hb) bound strongly to oxygen (O₂) and, chemically, the oxidation of Fe⁺⁺ to Fe⁺⁺⁺, for example. Methemoglobin (MetHb) may be produced in a body, by amyl nitrite (C₅H₁₁NO₂) or sulfamine (RSO₂NH₂) poisoning and may be produced outside the body when applying potassium ferritian or ozone (O₃) to blood, for example. Methemoglobin, unlike oxyhemoglobin, does not readily release oxygen and, thus, is not helpful for breathing.

In an embodiment, the signal acquirer 340 may select the lights of two or more wavelengths by a difference between the first absorption rate of carboxyhemoglobin (COHb), which is the target to be detected, and the second absorption rate of oxyhemoglobin and reduced hemoglobin, for example. The signal acquirer 340 may select the lights of two or more wavelengths, in which the second absorption rate is the same and the difference between the second absorption rate and the first absorption rate is greater than a predetermined standard, from among the reflected lights.

In an embodiment, when the absorption rate of oxyhemoglobin and reduced hemoglobin is the same as the second absorption rate, the signal acquirer 340 may select any one of a third wavelength band, in which the second absorption rate of the oxyhemoglobin and the reduced hemoglobin is different from a first absorption rate of a carboxyhemoglobin, and the first wavelength band or the second wavelength band, in which the second absorption rate is the same as the first absorption rate, from among the reflected lights, for example.

The signal acquirer 340 may determine a light of the first wavelength band or a light of the second wavelength band, in which the second absorption rate is substantially the same as the first absorption rate, as a light of a first wavelength among the reflected lights. The signal acquirer 340 may determine the light of the third wavelength band, in which the second absorption rate is not the same as the first absorption rate, as a light of a second wavelength. The signal acquirer 340 may calculate the first absorption rate corresponding to the target (e.g., carboxyhemoglobin (COHb)) of the light of the first wavelength and the light of the second wavelength. The signal acquirer 340, when the difference between the second absorption rate and the first absorption rate is greater than the predetermined standard, may select the light of the first wavelength and the light of the second wavelength.

In an embodiment, the first wavelength is about 520 nm, and the second wavelength is about 805 nm, for example.

More specifically, the signal acquirer 340 may select a light of wavelength(s), in which the absorption rate of the target (e.g., carboxyhemoglobin (COHb)) to be measured is significantly different, but the absorption rate of substances (e.g., oxyhemoglobin and reduced hemoglobin) other than the target to be measured is not changed and may acquire pulse wave signals.

In an embodiment, when the measurement target is carboxyhemoglobin (COHb), the signal acquirer 340 may be affected by the first absorption rate of carboxyhemoglobin (COHb) but may select any one wavelength pair of wavelength pairs (e.g., wavelengths of about 440 nm and about 805 nm, wavelengths of about 520 nm and about 805 nm, or wavelengths of about 440 nm and about 520 nm) from among wavelengths (e.g., about 440 nm, about 520 nm, and about 805 nm) not affected by the second absorption rate of oxyhemoglobin and reduced hemoglobin, for example. The signal acquirer 340 may select a pair of wavelengths of about 520 nm and about 805 nm, in which a difference between wavelengths is the greatest from among the wavelength pairs.

The signal acquirer 340 may acquire at least some of pulse wave signals corresponding to the selected wavelengths. The signal acquirer 340 may further include a reflective pulse sensor 345 and may acquire at least some of pulse wave signals through the reflective pulse sensor 345. In an embodiment, the signal acquirer 340 may continuously acquire pulse wave signals by setting sampling frequency to include a bandwidth (e.g., less than about 5 Hz) of a pulse wave signal, for example.

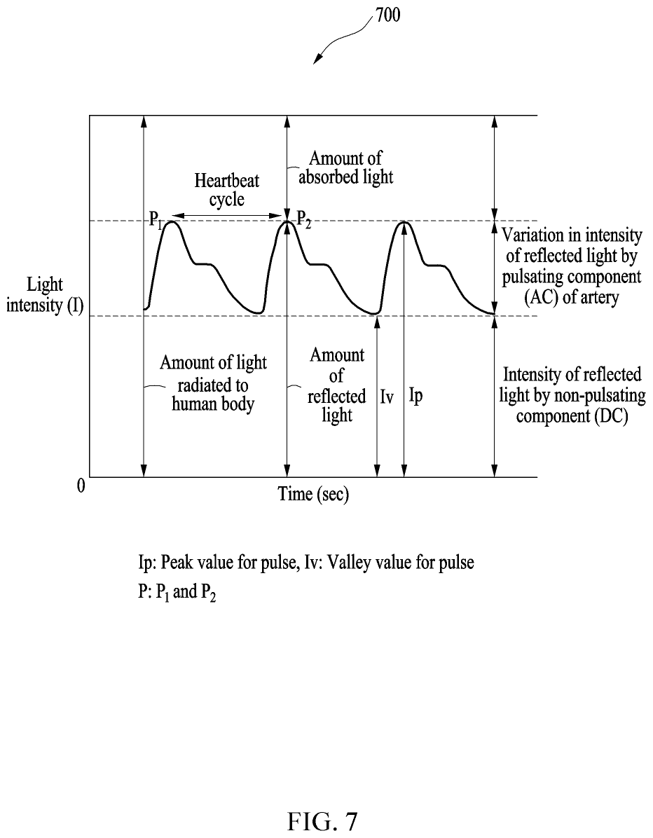

A ‘pulse wave’ may be commonly understood as photoelectric plethysmography. In an embodiment, the pulse wave signal may be detected by a volume change caused by a change in the amount of light reflected from or transmitted to the user's skin due to an increase or decrease of the amount of blood according to light absorption characteristics, such as the amount of light to be absorbed into hemoglobin in the blood in a wavelength range from visible light to near-IR light, for example. The measurement principle of pulse wave signals is described in detail below with reference to FIG. 7.

The estimator 350 may estimate a percentage of a target to be detected, based on a ratio of an absorption rate of each wavelength of the pulse wave signals acquired by the signal acquirer 340. In an embodiment, the estimator 350 may acquire pulse wave signals by each wavelength band of lights of two or more wavelengths and may calculate relative absorption rates by a volume change by each wavelength, based on the acquired pulse wave signals, for example. In an embodiment, the estimator 350 may find each point corresponding to contraction and relaxation from pulse wave signals acquired by each wavelength band and may calculate a relative absorption rate by volumetric conversion by each wavelength band by an optical signal when each point is found, for example.

The estimator 350 may estimate a percentage of the target of the total hemoglobin (Hb) including oxyhemoglobin and reduced hemoglobin, based on a ratio of an absorption rate of each wavelength of the pulse wave signals acquired by the signal acquirer 340.

The estimator 350 may estimate the percentage of the target by applying the ratio of the absorption rate of each wavelength to information corresponding to a pre-acquired target. In this case, the information corresponding to the target may include a difference between a pre-calculated ratio and a correlation coefficient of the concentration of the target. Here, the information corresponding to the target may correspond to information acquired through a clinical test stipulated by international organization for standardization (ISO), for example. As described above, since a pulse wave signal may be detected by a volume change caused by a change of the amount of light reflected from or transmitted to the user's skin caused by an increase or decrease of the amount of blood according to the absorption characteristics of a substance, in an embodiment, the percentage of the target may be estimated based on relative absorption rates by a volume change of each wavelength.

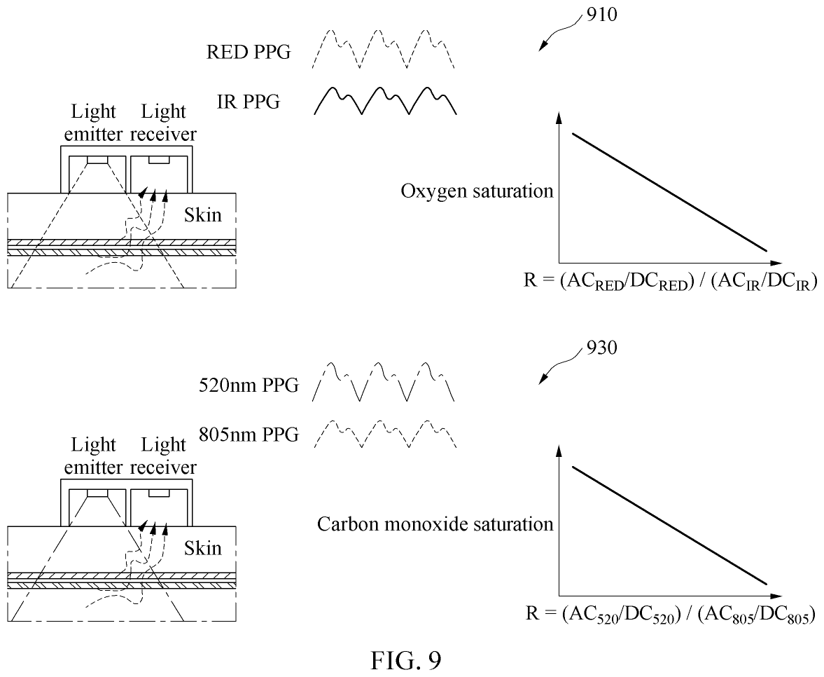

The wearable device 300 may define risk information, including mild, moderate, severe, and fatal, of the user depending on the size of the percentage, estimated by the estimator 350, of the target, for example. The principle of the estimator 350 estimating the percentage of the target to be detected is described in detail below with reference to FIG. 9.

The notifier 360 may provide risk information corresponding to the target through a notification, based on the estimated percentage of the target. The notifier 360 may notify the user of the risk information corresponding to the target in at least one notification method among the flickering of a screen (or display), the flashing of the screen, a haptic notification by touch (e.g., vibration), and notification sound, but examples are not limited thereto. In an alternative embodiment, the notifier 360 may notify a predetermined contact target of the risk information corresponding to the target through communication connection or message transmission to a predesignated contact. In this case, the predetermined contact target may be an external agency, such as an emergency medical agency, a fire station, or a police station, a family, an emergency contact target, or an external electronic device, for example.

In an alternative embodiment, the notifier 360 may display a message “Would you like to send your risk information notification?” on a screen, and, when the user's feedback, such as “No”, is not received within a few seconds, for example, may transmit a message notifying the risk information corresponding to the target to a contact target (the family or the emergency contact target) registered as the emergency contact target.

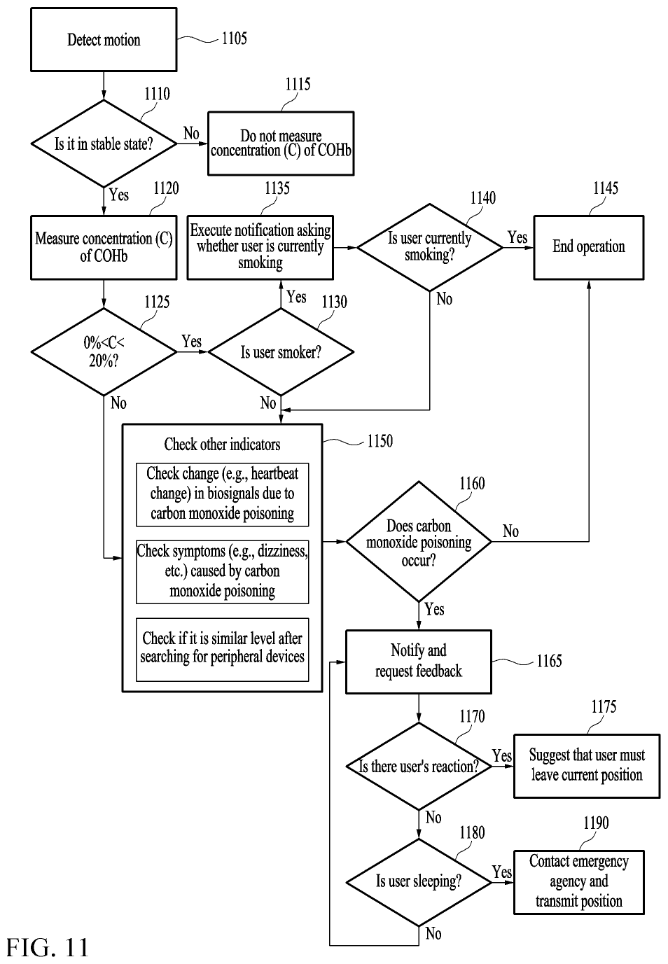

The wearable device 300 may determine whether the user is sleeping, based on the user's biometric information detected through sensors (not shown). The wearable device 300 may determine whether the user is sleeping by a commonly known method. The wearable device 300 may determine whether the user is sleeping by information acquired by a heart rate and PPG-based heart rate variability or the absence of movement of an accelerometer during a set time. When the user is determined to be sleeping, the wearable device 300 may determine whether the percentage of the target increases by a set threshold or more during a predetermined time interval, and, when the percentage of the target is determined to increase by the threshold or more, may notify the user, the external agency (e.g., the emergency medical agency), and/or the external electronic device of the user being in a risk state by the target through the notifier 360. The more predetermined operation of the notifier 360 is described in detail below with reference to FIG. 11.

The communication interface 370 may transmit the notification message generated in the notifier 360 to a rescue agency or may receive various pieces of data, such as the user's biosignal, from the outside of the wearable device 300. In addition, the communication interface 370 may transmit the percentage of the target estimated by the processor 305 to the outside of the wearable device 300.

The memory 380 may store a signal or data received through the communication interface 370 and/or the percentage of the target estimated by the processor 305.

The memory 380 may store various pieces of information generated in the processing of the signal acquirer 340, the estimator 350, the notifier 360, or the processor 305 described above. In addition, the memory 380 may store various pieces of data and programs. The memory 380 may include a volatile memory or a non-volatile memory. The memory 380 may include a high-capacity storage medium, such as a hard disk, to store the various pieces of data.

The display device 390 may display the percentage of the target estimated by the estimator 350. The display device 390 may be a touch display and/or a flexible display, for example, but examples are not limited thereto.

The processor 305 may execute a program and may control the wearable device 300. Program code to be executed by the processor 305 may be stored in the memory 380.

In addition, the processor 305 may perform at least one method to be described below with reference to FIGS. 4 to 11 or a scheme corresponding to the at least one method. The wearable device 300 may be a hardware-implemented electronic device having a circuit that is physically structured for the processor 305 to execute desired operations. In an embodiment, the desired operations may include code or instructions in a program, for example. The hardware-implemented processor 305 may include a microprocessor, a central processing unit (CPU), a GPU, a processor core, a multi-core processor, a multiprocessor, an application-specific integrated circuit (ASIC), a field-programmable gate array (FPGA), and/or a neural processing unit (NPU), for example.

FIG. 4 is a diagram illustrating an embodiment of the structures of a light emitter and a light receiver. FIG. 4 includes diagram 401 illustrating a wearable device (e.g., the wearable device 100 of FIG. 1, the wearable device 200 of FIG. 2, and/or the wearable device 300 of FIG. 3) in an embodiment including one light emitter 410 (e.g., the light emitter 310 of FIG. 3 and/or the light emitter 510 of FIG. 5) and one light receiver 460 (e.g., the light emitter 320 of FIG. 3), diagram 403 illustrating the wearable device 300 including a plurality of light emitters 410, 420, and 430 and one light receiver 460, and diagram 405 illustrating the wearable device 300 including one light emitter 440 including a multispectral light source and one light receiver 460. In this case, the analog circuit & ADC 330 may perform signal processing including signal amplification and filtering for reflected lights detected by the light receiver 460 and may convert a signal-processed reflected lights into digital signals.

In an embodiment, referring to diagram 401, the wearable device 300 may include the light emitter 410 that emits a light of a single wavelength band by one LED light source and one light receiver 460 that detects reflected lights reflected at least partially from a user's skin by the single wavelength band, for example.

In an alternative embodiment, as shown in diagram 403, the wearable device 300 may include multiple light emitters that generate lights of respective single wavelength bands. In this case, each of the plurality of light emitters 410, 420, and 430 may include multiple LED light sources that generate lights of different wavelengths. In an embodiment, the light emitter 410 may include a green light LED that generates a light of about 530 nm wavelength as a light source, for example. The light emitter 420 may include a red-light LED that generates a light of about 660 nm wavelength as a light source. In addition, the light emitter 430 may include an IR-light LED that generates a light of about 940 nm wavelength as a light source.

Referring to diagram 405, the wearable device 300 may include the light emitter 440 including a multispectral light source that generates multiple wavelengths within a spectral sensor. In this case, the spectral sensor may be a sensor that creates a spectrum by dispersing light radiation from one light source (e.g., a laser light source) and quantitatively measures radiation intensity in the position of various wavelengths of the spectrum.

In embodiments, the wearable device 300 may also include a single light emitter and multiple light receivers. The embodiment of the wearable device including a single light emitter and multiple light receivers is described below with reference to FIG. 5.

FIG. 5 is a diagram illustrating an embodiment of an arrangement relationship between a light emitter and a light receiver of the wearable device. FIG. 5 includes diagram 500 illustrating the arrangement relationship between a light emitter 510 (e.g., the light emitter 310 of FIG. 3 and/or the light emitters 410, 420, 430, and 440 of FIG. 4) and a plurality of light receivers 530 (e.g., the light receiver 320 of FIG. 3 and/or the light receiver 460 of FIG. 4) on the second surface 110B of the wearable device (e.g., the wearable device 100 of FIG. 1, the wearable device 200 of FIG. 2, and/or the wearable device 300 of FIG. 3) in an embodiment.

Although FIG. 5 illustrates an embodiment of the light emitter 510 being a single light emitter and the plurality of light receivers 530 being multiple light receivers, examples are not limited thereto. The arrangement relationship may also apply to a single light emitter and a single light receiver, multiple light emitters and a single light receiver, and multiple light emitters and multiple light receivers.

A light source (e.g., the light source 315 of FIG. 3) of the light emitter 510 may be in the center of the second surface 110B of the wearable device 300, for example. The plurality of light receivers 530 may be radially around the light source of the single light emitter 510.

The plurality of light receivers 530 may be within a distance or a range to which sufficient light that has been emitted from the light emitter 510 and backscattered reaches, for example. Here, the ‘sufficient light’ may be understood as the degree (size) of a light signal component where a pulse wave signal may be measured.

In an embodiment, an arrangement distance between light receiver(s) and light emitter(s) may vary depending on wavelength bands of light. In an embodiment, when a light source of light emitter(s) is an about 805 nm IR light source with a relatively high light penetration depth into a human tissue, for example, the light receiver(s) may be apart relatively farther from the light emitter(s) than when the light source is a light source with a relatively low light penetration depth. In contrast, when the light source of the light emitter(s) is an about 440 nm or 520 nm red light source with a relatively low light penetration depth, the light emitter(s) may be relatively closer to the light receiver(s) than when the light source is a light source with a relatively high light penetration depth.

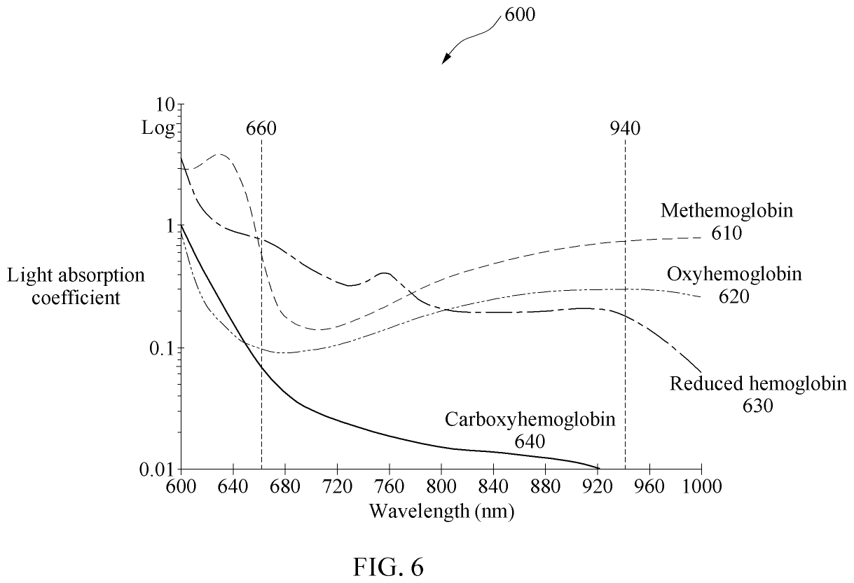

FIG. 6 is a diagram illustrating absorption spectra of hemoglobin in various states by a plurality of lights of wavelengths. FIG. 6 includes graph 600 illustrating absorption spectra corresponding respectively to extinction coefficients of a methemoglobin (MetHb) 610, oxyhemoglobin 620, a reduced hemoglobin 630, and a carboxyhemoglobin 640 in a 660 nm red-light wavelength to a 940 nm IR wavelength.

In an embodiment, pulse oximetry may measure oxygen saturation (SpO₂) noninvasively through a skin part of fingertip, wrist, earlobe, or forehead based on a pulse wave of PPG by the 660 nm red-light wavelength to the 940 nm IR wavelength, for example. The oxygen saturation (SpO₂) may be an indicator that digitizes an amount of hemoglobin bound to oxygen in the blood as a percentage.

Such oxygen saturation measured through a skin part may be also referred to as saturation of percutaneous oxygen (SpO₂). The pulse oximetry that measures the saturation of percutaneous oxygen (SpO₂) based on a pulse wave of PPG may not readily detect the carboxyhemoglobin (COHb) 640. This is because of the optical properties of the carboxyhemoglobin (COHb) 640 where the carboxyhemoglobin (COHb) 640 affects an absorption rate of a red light of a 660 nm wavelength selectively.

In an embodiment, when a person has 100% oxygen saturation, blood in their body may be the oxyhemoglobin 620. Thus, the ratio of light absorption rates in 660 nm and 940 nm wavelengths may be about 0.11 divided by about 0.55 (i.e., about 0.11/about 0.55=about 0.2), for example. When a person has 0% oxygen saturation, blood in their body may be the reduced hemoglobin 630. Thus, the ratio of light absorption rates in 660 nm and 940 nm wavelengths may be about 0.84 divided by about 0.3 (i.e., about 0.84/about 0.3=about 28), for example. The ratio of oxygenated hemoglobin of the hemoglobin may be calculated as a difference between the ratios 0.2 and 28.

However, referring to graph 600, as the concentration of carboxyhemoglobin (COHb) 640 increases, only the oxyhemoglobin 620 may be absorbed additionally. Thus, an R value corresponding to a ratio of the size of a pulse wave signal in a red-light wavelength (660 nm)/the size of a pulse wave signal in an IR wavelength (940 nm) may decrease. Here, the R value may be R of a modulation ratio of a Monte Carlo (MC) model. The description thereof is described in more detail below with reference to FIG. 9. Since the decrease of the R value leads to the increase of the percutaneous oxygen concentration (SpO₂), oxygen saturation may be measured falsely as relatively high in the case of carbon monoxide poisoning.

FIG. 7 is a diagram illustrating an embodiment of the intensity of the light absorbed into and reflected from a human tissue. FIG. 7 includes graph 700 illustrating an embodiment of a light intensity I changing by a pulsation component of arterial blood according to a time (sec).

The heart may contract and relax through the sinoatrial node in the atrium that determines a rhythm throughout the heartbeat. Blood ejected from the left ventricle during systole may move to peripheral blood vessels and the volume of blood vessels in arteries may increase. A pulse may be generated by partial suction from peripheral blood vessels to the heart during diastole. When measuring the intensity of light reflected from the skin by irradiating light on the generated pulse, a signal that moves periodically according to heartbeats may be acquired.

In an embodiment, referring to graph 700, when the maximum peak point of a waveform is denoted by the maximum systole P of heart contraction, a period from a first maximum systole P₁ to a second maximum systole P₂ may correspond to a heartbeat cycle, for example. The heartbeat cycle may be also referred to as a ‘pulse’.

Graph 700 represents a relationship between the amount of light irradiated to the body and the amount of light absorbed by the body through a pulse waveform changing over time. In this case, a non-pulsating component by static blood flow may be expressed by ‘DC’ and a pulsating component by pulsatile blood flow may be expressed by ‘AC’. A variation in the intensity of reflected light by the pulsating component (AC) of the artery may be caused mainly by a pulse waveform implemented in a variation of blood flow by heartbeat and a waveform having a weak signal for heartbeat. Here, the waveform having a weak signal for heartbeat may be generated by breathing or motion of a person, for example, but examples are not limited thereto. In addition, the intensity of reflected light by the non-pulsating component (DC) may be affected by light being absorbed or scattered by body components, such as bone, skin, or subcutaneous tissue, which do not change over time.

In an embodiment, blood oxygen saturation may be measured by the Beer-Lambert law, for example. According to the Beer-Lambert law, an absorption rate at a given wavelength may be proportional to the absorption rate of a sample of a light path region passed by a light emitted from a light emitter and received by a light receiver, a path length, and the concentration of the absorbed sample. Based on the Beer-Lambert law, oxygen saturation may be measured by measuring the ratio of absorption rates of two wavelengths reflected after irradiating light having two different wavelengths to body tissue.

According to the Beer-Lambert law, the amount of light absorbed by body tissue may be calculated by a ratio of the intensity of reflected light to the intensity of incident light irradiated to the body at a predetermined wavelength by a light source. However, it is difficult to explain reflectivity on the skin's surface or scattering effect in body tissues with the Beer-Lambert law. Thus, to these phenomena, photon diffusion theory may be applied.

Oxygen saturation in the blood may be measured by absorption rates by each of wavelengths of two different incident lights transmitted to and reflected from a tissue, for example. In this case, the wavelengths of different incident lights may be an IR light of about 940 nm wavelength and a red light of about 660 nm wavelength where a difference between absorption rates of hemoglobin and oxyhemoglobin is large, for example.

In an embodiment, oxygen saturation may be expressed by a percentage of oxyhemoglobin (HbO₂) to the sum of oxyhemoglobin (HbO₂) and hemoglobin (Hb) in the blood, for example, as shown in Equation 1.

$$Oxygen Saturation ( \% ) = \frac{{Hb O}_{2}}{{Hb O}_{2} + Hb} \times 100 \qquad [ Equation 1 ]$$

The above Equation 1 is just an example to aid understanding, but the calculation method of the percentage of hemoglobin (HbO₂) is not limited thereto and may be modified, applied, or expanded in various manners.

In addition, percutaneous oxygen saturation (SpO₂) may be obtained through the Beer-Lambert law and photon diffusion theory as shown in Equation 2 below.

$${S}_{p} {O}_{2} = X + Y Φ \qquad [ Equation 2 ]$$

The above Equation 2 is just an example to aid understanding, but the calculation method of the percutaneous oxygen saturation (SpO₂) is not limited thereto and may be modified, applied, or expanded in various manners.

Here, X denotes an optical characteristic constant of hemoglobin (Hb) and Y denotes an optical characteristic constant of oxyhemoglobin (HbO₂). In addition, Φ denotes a ratio of absorption rates A of hemoglobin (Hb) and oxyhemoglobin (HbO₂) for each of a red light of about 660 nm wavelength and an IR light of about 940 nm wavelength, which is A₆₆₀ₙₘ/A₉₄₀ₙₘ.

Percutaneous oxygen saturation (SpO₂) may be detected through different absorption characteristics by each wavelength band when the red light of about 660 nm wavelength and the IR light of about 940 nm wavelength are irradiated to body tissue.

In an embodiment, the blood concentration of carboxyhemoglobin (COHb) may be calculated by similarly applying the Beer-Lambert law and photon diffusion theory described above.

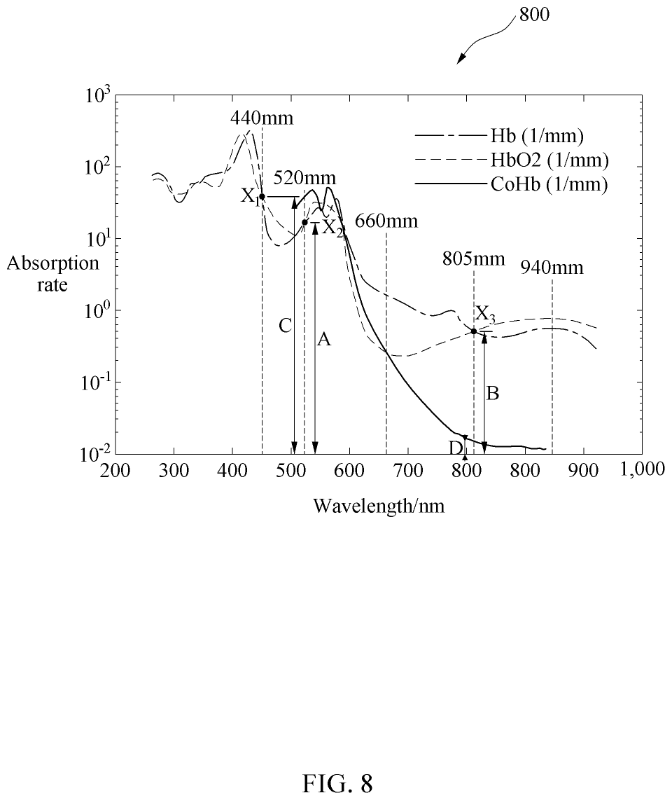

FIG. 8 is a diagram illustrating an embodiment of the principle of detecting blood carboxyhemoglobin (COHb). FIG. 8 includes graph 800 illustrating light absorption rates by wavelength bands of oxyhemoglobin (HbO₂) bound to oxygen, oxyhemoglobin (HbO₂) unbound to oxygen, and oxyhemoglobin (HbO₂) bound to carbon monoxide.

There may be a difference between oxyhemoglobin (HbO₂) and hemoglobin (Hb) in light absorption rates. In an embodiment, for a red light of about 660 nm wavelength, there may be a relatively large difference in light absorption rates of oxyhemoglobin (HbO₂) and hemoglobin (Hb), for example. The light absorption rate of hemoglobin may be higher than the light absorption rate of oxyhemoglobin in a red-light region (660 nm). There may be a relatively smaller difference in the light absorption rates of oxyhemoglobin (HbO2) and hemoglobin (Hb) for an IR light of about 830 nm wavelength than a difference in the light absorption rates of oxyhemoglobin (HbO2) and hemoglobin (Hb) for the red light of about 660 nm wavelength. In addition, the light absorption rate of oxyhemoglobin may be higher than the light absorption rate of hemoglobin in a far-IR light of about 940 nm wavelength.

The critical reason why it has been difficult for IR- and red light-based pulse oximetry to detect the presence of carboxyhemoglobin (COHb) is that both an IR-based pulse wave signal and a red light-based pulse wave signal are affected by oxyhemoglobin (HbO₂) and hemoglobin (Hb).

In an embodiment, when selecting two wavelengths irrelevant to the oxidation or deoxidation (e.g., reduction) of hemoglobin (Hb) by oxygen and measuring percutaneous oxygen saturation (SpO₂) through the skin by a pulse wave signal acquired from the selected two wavelengths, absorption rates may have substantially the same ratio regardless of oxygen saturation, for example. In an embodiment, since a pulse wave signal is acquired by wavelengths where absorption rates have the same ratio, two wavelengths having the same absorption rate may be selected regardless of oxidation or reduction.

Referring to graph 800, wavelengths with absorption rates having substantially the same ratio may be intersection point(s) X₁, X₂, or X₃ of two spectra corresponding to oxyhemoglobin (HbO₂) and hemoglobin (Hb). The intersection point X₁ may appear at an about 440 nm wavelength, the intersection point X₂ may appear at an about 520 nm wavelength, and the intersection point X₃ may appear at an about 805 nm wavelength.

Referring to graph 800, when carboxyhemoglobin (COHb) is not present, and pulse wave signals acquired by the about 520 nm wavelength and the about 805 nm wavelength are used, for example, perfusion indices (PIs) acquired respectively from the about 520 nm wavelength and the about 805 nm wavelength may represent an A/B ratio.

Referring to graph 800, A denotes a light absorption coefficient of HbO₂ at about 520 nm and B denotes alight absorption coefficient of HbO₂ at about 805 nm. C denotes a light absorption coefficient of COHb at 520 nm and D denotes a light absorption coefficient of COHb at 805 nm.

In this case, the A/B ratio may not be affected by an oxygen saturation level. A pulse intensity index (PI) may be a quantitative indicator that indicates hemodynamic stability by evaluating the pulse wave intensity of a peripheral blood vessel. The pulse intensity index (PI) may be a relative indicator of a light absorption rate as a pulsating component (‘AC component’) by pulsatile blood flow/a non-pulsating component (‘DC component’) by static blood flow. The pulse intensity index (PI) may also be also referred to as a ‘perfusion index’.

For extreme example, when only carboxyhemoglobin (COHb) is in the blood, the carboxyhemoglobin (COHb) may not be affected by the concentration of oxyhemoglobin (HbO₂) and hemoglobin (Hb) at about 440 nm, 520 nm, and 805 nm wavelengths, and the pulse intensity index (PI) may change from A/B to C/D. Practically, the carboxyhemoglobin (COHb) in the blood may have any ratio between A/B to C/D.

The wearable device (e.g., the wearable device 100 of FIG. 1, the wearable device 200 of FIG. 2, and/or the wearable device 300 of FIG. 3) in an embodiment may identify different concentration gradients of the carboxyhemoglobin (COHb) by each wavelength band by a ratio gradient between A/B to C/D. The wearable device 300 may calculate the concentration of carbon monoxide in the air and the ratio of the carboxyhemoglobin (COHb) in the blood through calibration.

In an embodiment carboxyhemoglobin (COHb) may be detected by a pair of two wavelengths among wavelengths, such as about 440 nm, 520 nm, and 805 nm wavelengths, where oxyhemoglobin (HbO₂) intersects with hemoglobin (Hb), for example.

In addition, in an embodiment, a wavelength corresponding to a green light that may acquire a stable heart rate during exercise may be further used to detect carboxyhemoglobin (COHb), other than an IR light and a red light included in the wearable device 300 to measure oxygen saturation.

FIG. 9 is a diagram illustrating an embodiment of the principle of estimating a percentage of a target to be detected by an estimator. FIG. 9 includes diagram 910 illustrating the operating principle of pulse oximetry, which is an oxygen saturation measuring sensor relatively showing an amount of oxygen dissolved in the blood, according to a comparative embodiment, and diagram 930 illustrating an embodiment of the operating principle of an estimator (e.g., the estimator 350 of FIG. 3) of a wearable device (e.g., the wearable device 100 of FIG. 1, the wearable device 200 of FIG. 2, and/or the wearable device 300 of FIG. 3), in an embodiment.

As described above, the reflectivity and intensity of lights of different wavelengths transmitted through vessels may differ depending on the movement of the blood and/or a change in pressure, for example. The difference in the reflectivity of the lights by a light source (e.g., the light source 315 of FIG. 3) may be reflected on reflected lights detected in a receiver (e.g., the receiver 320 of FIG. 3, the receiver 460 of FIG. 4, and/or the receiver 530 of FIG. 5), such as a photodiode.

In an embodiment, referring to diagram 910, light irradiated to a fingertip by pulse oximetry is absorbed by tissues, veins, and the blood, and an absorption rate may increase, for example. When arteries are empty, the absorption rate may decrease. Thus, the absorption rate by the blood may be expressed by a pulsating component (AC component). In an embodiment, the remaining elements, such as skin and tissues, excluding the blood may be non-pulsating components (DC components), for example. The non-pulsating components (DC components) may be base values.

The pulse oximetry generally uses two LEDs, e.g., a red-light LED and an IR light LED, as light sources to irradiate light and may use an absorption rate of a tissue for the irradiated light to measure the oxygen saturation of the blood. In this case, a wavelength of the irradiated light may be selected based on relative light absorption coefficients of oxygenated hemoglobin (HbO₂) and oxygen-free hemoglobin (Hb). In an embodiment, an MC model or a photon diffusion (PD) model may be used to analyze a difference between a red-light wavelength and an IR light wavelength, for example. The MC model may use a probability between the sizes of signals. A modulation ratio R of the MC model may be obtained by, a ratio between absorption coefficients of body tissues during systole and diastole, for example.

The modulation ratio R may be the size of a pulse wave signal corresponding to a red-light wavelength/the size of a pulse wave signal corresponding to an IR light wavelength, for example, as shown in Equation 3 below.

$$R = ( {AC}_{RED} / {DC}_{RED} ) / ( {AC}_{IR} / {DC}_{IR} ) \qquad [ Equation 3 ]$$

The above Equation 3 is just an example to aid understanding, but the calculation method of the modulation ratio R is not limited thereto and may be modified, applied, or expanded in various manners.

In an embodiment, the estimator 350 uses a similar principle of the pulse oximetry but different wavelengths from wavelengths of the pulse oximetry and may estimate a percentage of carboxyhemoglobin (COHb).

Referring to diagram 930, the estimator 350 may estimate the percentage of carboxyhemoglobin (COHb) by a ratio R of the size of a pulse wave signal corresponding to an about 520 nm wavelength/the size of a pulse wave signal corresponding to an about 805 nm wavelength.

In an embodiment, the ratio R of the size of the pulse wave signal corresponding to an about 520 nm wavelength/the size of the pulse wave signal corresponding to an about 805 nm wavelength, for example, may be expressed by Equation 4 below.

$$R = ( {AC}_{520} / {DC}_{520} ) / ( {AC}_{805} / {DC}_{805} ) \qquad [ Equation 4 ]$$

The above Equation 4 is just an example to aid understanding, but the calculation method is not limited thereto and may be modified, applied, or expanded in various manners.

The estimator 350 may find a first point corresponding to contraction and a second point corresponding to relaxation in pulse wave signals acquired by each wavelength band and may calculate relative absorption rates by a volumetric change by each wavelength by an absorption rate difference of light signals corresponding to the first point and the second point.

The estimator 350 may calculate a ratio of the absorption rates by each wavelength from pulse wave signals corresponding to two wavelengths. The estimator 350 may estimate the percentage (%) of carboxyhemoglobin (COHb) by substituting the calculated ratio of the absorption rates by each wavelength into a correlation coefficient of (pre-calculated ratio-concentration of carboxyhemoglobin (COHb)). Here, the “correlation coefficient of pre-calculated ratio-concentration of carboxyhemoglobin (COHb)” may be obtained through a clinical test stipulated by ISO, for example.

In an embodiment, it may be assumed that there is no carboxyhemoglobin (COHb) in the blood, all the hemoglobin (Hb) is in the form of oxyhemoglobin (HbO₂) or hemoglobin (Hb), for example. In this case, the ratio of absorption rates measured at about 520 nm wavelength/about 805 nm wavelength may be calculated as A/B.

In contrast, it may be assumed that all the hemoglobin (Hb) in the blood is only in the form of carboxyhemoglobin (COHb). In this case, the ratio of absorption rates measured at about 520 nm wavelength/about 805 nm wavelength may be changed to C/D greater than A/B.

The ratio of absorption rates may have a value between A/B and C/D depending on the proportion of carboxyhemoglobin (COHb) present in hemoglobin (Hb). In this case, by reference equipment (e.g., a blood gas analyzer known as CO-Oximetry), the ground truth that approximately what % of carboxyhemoglobin (COHb) is present under each condition may be measured. When repeating this for several points, a relation of the percentage (%) of carboxyhemoglobin (COHb) to a pair of ratios of absorption rates measured at about 520 nm wavelength/about 805 nm wavelength may be obtained. The estimator 350 may estimate the percentage (%) of carboxyhemoglobin (COHb) by the relation obtained through said process. Here, ‘several points’ may refer to several points for the percentage (%) of carboxyhemoglobin (COHb), that is, different actual values. The ‘repeating this for several points’ may be understood as repeating the calculation of the ratio of absorption rates within a range of 0, 5, 10, 15, 20, and 25 percentages (%) of carboxyhemoglobin (COHb), for example.

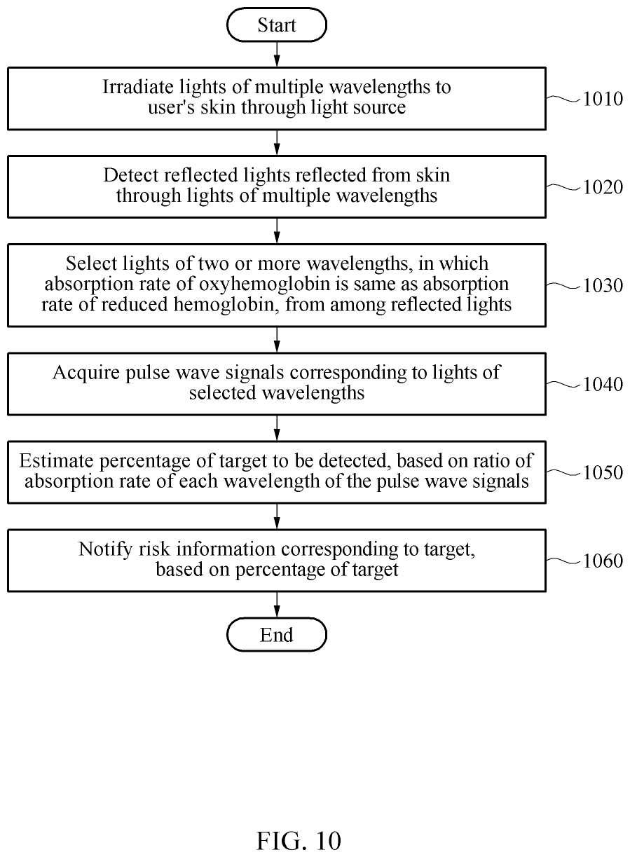

FIG. 10 is a flowchart illustrating an embodiment of an operating method of the wearable device. In the following embodiments, operations may be performed sequentially but may not be necessarily performed sequentially. In an embodiment, the order of the operations may be changed and at least two of the operations may be performed in parallel, for example.

In an embodiment, operations 1010 to 1060 may be understood as performed in a processor (e.g., the processor 305 of FIG. 3) of a wearable device (e.g., the wearable device 100 of FIG. 1, the wearable device 200 of FIG. 2, and/or the wearable device 300 of FIG. 3).

Referring to FIG. 10, in an embodiment, the wearable device may notify risk information by estimating a percentage of a target to be detected through operations 1010 to 1060.

In operation 1010, the wearable device 300 may irradiate lights of multiple wavelengths to a user's skin through a light source. In an embodiment, the wearable device 300 may irradiate the lights of multiple wavelengths on the user's skin through a light emitter (e.g., the light emitter 310 of FIG. 3, the light emitters 410, 420, and 430 of FIG. 4, or the light emitter 510 of FIG. 5) including the light source (e.g., the light source 315 of FIG. 3) that emits the lights of a first wavelength band (about 540 nm) corresponding to a green light, a second wavelength band (about 660 nm) corresponding to a red light, and a third wavelength band (about 940 nm) corresponding to an IR light, for example. In an embodiment, the light source 315 may be one LED light source that emits a light of a single wavelength band, multiple LED light sources that emit a light of a single wavelength band, and a multispectral light source that emits multiple wavelengths in a spectral sensor, for example, but examples are not limited thereto. The light emitter may be a single light emitter and a plurality of light emitters. In an embodiment, when the number of light emitters is plural, the wearable device 300 may divide time and may operate multiple light emitters (e.g., the light emitters 410, 420, and 430 of FIG. 4) corresponding to different wavelengths at regular time intervals, for example. The wearable device 300 may acquire pulse wave signals by reflected lights detected by a light receiver (e.g., the light receiver 320 of FIG. 3, the light receiver 460 of FIG. 4, and/or the light receiver 530 of FIG. 5) based on the operation of the light emitters 410, 420, and 430.

In operation 1020, the wearable device 300 may detect reflected lights reflected from the skin through the lights of multiple wavelengths irradiated in operation 1010.

In operation 1030, the wearable device 300 may select lights of two or more wavelengths, in which the absorption rate of oxyhemoglobin is the same as the absorption rate of a reduced hemoglobin, from among the reflected lights detected in operation 1020.

The wearable device 300 may select the lights of two or more wavelengths by a difference between a second absorption rate corresponding to oxyhemoglobin and reduced hemoglobin and a first absorption rate of the target to be detected.

The wearable device 300 may select the lights of two or more wavelengths, in which the second absorption rate is the same and the difference between the second absorption rate and the first absorption rate is greater than a predetermined standard, from among the reflected lights detected in operation 1020.

In operation 1040, the wearable device 300 may acquire pulse wave signals corresponding to the lights of wavelengths selected in operation 1030.

In operation 1050, the wearable device 300 may estimate a percentage of a target to be detected, based on a ratio of the absorption rate of each wavelength of the pulse wave signals acquired in operation 1040. In an embodiment, the wearable device 300 may calculate relative absorption rates by a volume change by each wavelength, based on the pulse wave signals acquired in operation 1040, and may estimate the percentage of the target by applying the relative absorption rates to pre-acquired information corresponding to the target, for example. In this case, the information corresponding to the target may include a difference between a pre-calculated ratio and a correlation coefficient of the concentration of the target.

The wearable device 300 may estimate a percentage of the target of the total hemoglobin (Hb) including oxyhemoglobin and reduced hemoglobin, based on a ratio of the absorption rate of each wavelength of the pulse wave signals acquired in operation 1040.

In operation 1060, the wearable device 300 may notify risk information corresponding to the target, based on the percentage of the target estimated in operation 1050. The wearable device 300 may define risk information, including mild, moderate, severe, and fatal, of the user depending on the size of the percentage of the target estimated in operation 1050, for example, but examples are not limited thereto.

FIG. 11 is a flowchart illustrating an embodiment of the operating method of the wearable device. In the following embodiments, operations may be performed sequentially but may not be necessarily performed sequentially. In an embodiment, the order of the operations may be changed and at least two of the operations may be performed in parallel, for example.

In an embodiment, operations 1105 to 1190 may be understood as performed in a processor (e.g., the processor 305 of FIG. 3) of a wearable device (e.g., the wearable device 100 of FIG. 1, the wearable device 200 of FIG. 2, and/or the wearable device 300 of FIG. 3).

Referring to FIG. 11, in an embodiment, the wearable device may perform operations 1105 to 1190.

In operation 1105, the wearable device 300 may detect a user's motion. In an embodiment, the wearable device 300 may detect the user's motion by any one or a combination of pulse wave signals detected in an acceleration sensor, a motion sensor, a gyro sensor, and/or a pulse sensor (e.g., the reflective pulse sensor 345 of FIG. 3), for example.

In operation 1110, the wearable device 300 may determine whether the user is in a stable state, based on the result of detection in operation 1105. In an embodiment, the ‘stable state’ may be understood as a state of no mobility, such as the user's state of sitting, standing, lying, or sleeping, other than an active state in which the user is moving or performing vigorous exercise, for example.

When the user is determined to be not in the stable state in operation 1110 (No), in other words, when the user is in the active state, the wearable device 300 may end the operation without measuring concentration C of carboxyhemoglobin (COHb) in the user's blood in operation 1115.

When the user is determined to be in the stable state in operation 1110, the wearable device 300 may measure the concentration C of carboxyhemoglobin (COHb) in the user's blood in operation 1120. Here, the ‘measuring the concentration C of carboxyhemoglobin (COHb) in the blood’ may be understood as including ‘estimating the percentage of carboxyhemoglobin (COHb)’.

In operation 1125, the wearable device 300 may determine whether the concentration C of carboxyhemoglobin (COHb) measured in operation 1120 is greater than about 0% (or about 5%) and less than about 20%, for example. In an embodiment, when the user is a smoker, the concentration C of carboxyhemoglobin (COHb) may be a value within about 20%, for example. When the concentration C of carboxyhemoglobin (COHb) is determined to be greater than about 0% (or about 5%) and less than about 20%, the wearable device 300 may determine the user is a smoker in operation 1130. In operation 1130, the wearable device 300 may ask the user whether he or she is a smoker through a user interface (UI) displayed in a display device (e.g., the display 220 of FIG. 2 or the display device 390 of FIG. 3) and may determine that the user is a smoker based on the user's response thereto or may determine whether the user is a smoker based on pre-stored initial user information, for example. The initial user information may include the user's weight, height, pressure, smoking status, alcohol consumption, and/or chronic illness, for example, but examples are not limited thereto.

In operation 1130, when the user is determined to be a smoker, the wearable device 300 may execute a notification asking whether the user is currently smoking in operation 1135.

In operation 1130, when the user is determined to be not a smoker, the wearable device 300 may perform operation 1150 described below.

Even when the concentration (C) of carboxyhemoglobin (COHb) is determined to be less than about 0% (or about 5%) or greater than about 20% in operation 1125, the wearable device (300) may perform operation 1150 described below.

According to the execution of the notification in operation 1135, the wearable device 300 may ask whether the user is currently smoking through a pop-up message in operation 1140, for example.

When the user answers that he or she is currently smoking to the question in operation 1140 (Yes), the wearable device 300 may end the operation by ignoring the concentration C of carboxyhemoglobin (COHb) measured above in operation 1145.

When the user answers that he or she is currently not smoking to the question in operation 1140 (No), the wearable device 300 may check other indicators in operation 1150. By checking other indicator(s), other than the concentration C of carboxyhemoglobin (COHb), operation 1150 may be an operation for improving the detection accuracy of carbon monoxide poisoning.

In operation 1150, the wearable device 300 may check a change (e.g., heart rate change) of a biosignal due to carbon monoxide poisoning or may check or ask the user on various symptoms (e.g., headache, dizziness, nausea, seizures, and/or respiratory paralysis) caused by carbon monoxide poisoning, for example. In addition, the wearable device 300 may check whether the concentration of hemoglobin detected after searching for peripheral devices (e.g., medical devices, Internet-of-Things (IoT) devices, or other sensors) in a similar manner to short-range communication (e.g., Bluetooth) is similar to a value measured by the wearable device 300 or a similar pattern is observed in the peripheral devices.

Based on remaining (the other) indicators checked in operation 1150, the wearable device 300 may determine whether carbon monoxide poisoning occurs in operation 1160.

When carbon monoxide poisoning is determined to not occur in operation 1160 (No), the wearable device 300 may end the operation by ignoring the concentration C of carboxyhemoglobin (COHb) measured above in operation 1145.

In contrast, when carbon monoxide poisoning is determined to occur in operation 1160 (Yes), the wearable device 300 may provide a notification warning to the user and may request feedback on the notification from the user in operation 1165. In an embodiment, the wearable device 300 may provide the notification through warning sound, vibration, and/or flickering signals, for example, but examples are not limited thereto.

Regarding the notification and feedback request provided in operation 1165, the wearable device 300 may determine whether there is the user's reaction (e.g., motion) in operation 1170.

When it is determined that there is the user's reaction in operation 1170 (Yes), the wearable device 300 may suggest that the user must leave the current position in operation 1175.

When it is determined that there is no user's reaction in operation 1170 (No), the wearable device 300 may determine whether the user is sleeping based on the user's biosignals in operation 1180.

When the user is determined to be not sleeping in operation 1180, the wearable device 300 may perform operation 1165 for providing a notification warning to the user and requesting feedback on the notification from the user.

When the user is determined to be sleeping in operation 1180, the wearable device 300 may transmit the user's position by contacting an emergency agency while notifying the emergency agency of the user being in a poisoned state caused by carboxyhemoglobin (COHb) in operation 1190.

When the user is determined to be sleeping in operation 1180, the user is defenseless where self-action is impossible. Thus, the wearable device 300 may determine whether the concentration C of carboxyhemoglobin (COHb) increases gradually or whether the concentration C of carboxyhemoglobin (COHb) decreases gradually. In an embodiment, when the concentration C of carboxyhemoglobin (COHb) is less than a predetermined numerical value (e.g., about 20%), the wearable device 300 may attempt to wake up the user in a sleep state by vibration and/or sound, for example.

In an embodiment, when the concentration C of carboxyhemoglobin (COHb) increases gradually and the percentage of carboxyhemoglobin (COHb) increases by a threshold or more during a predetermined time interval, the wearable device 300 may transmit information notifying carbon monoxide poisoning is happening and prompt rescue is desired to an emergency agency together or may make urgent contact automatically with registered acquaintances, for example.

In an embodiment, the wearable device 300 may perform on-demand measurement of carbon monoxide poisoning or may perform monitoring through continuous or periodical measurement.

In an embodiment, when the on-demand measurement of carbon monoxide poisoning is performed, and the wearable device 300 detects a change in a biosignal caused by carbon monoxide poisoning or the user recognizes carbon monoxide poisoning symptoms and notifies the symptoms, the percentage of carboxyhemoglobin (COHb) may be measured on demand, for example. The wearable device 300 may provide appropriate guidance while improving the accuracy of detecting carbon monoxide poisoning through the percentage of carboxyhemoglobin (COHb), questionnaire information requested additionally to the user, and other indicators.

When monitoring carbon monoxide poisoning, the wearable device 300 may check whether the percentage of carboxyhemoglobin (COHb) increases gradually or decreases gradually through continuous estimation.

In an embodiment, to determine carbon monoxide poisoning during sleep, the wearable device 300 may determine whether the percentage of carboxyhemoglobin (COHb) increases by a predetermined value (e.g., about 3 to 4%) or more during a predetermined window time interval (e.g., about 1 minute), for example. In this case, the ‘specific value’ may vary depending on whether the user is a smoker or not. In an embodiment, when the user is a smoker, the wearable device 300 may define that the percentage of the carboxyhemoglobin (COHb) of the user increasing by about 3% to 4% as carbon monoxide poisoning, for example. When the user is a non-smoker, the wearable device 300 may define that the percentage of the carboxyhemoglobin (COHb) of the user increasing by about 10% as carbon monoxide poisoning. The average carbon monoxide level in the blood of smokers may be generally higher than the average carbon monoxide level in the blood of non-smokers. Therefore, to reduce false positives where something false is determined falsely to be true, in an embodiment, the level of the percentage of carboxyhemoglobin (COHb) for determining the carbon monoxide poisoning of smokers may be set higher than that of non-smokers. The wearable device 300 may set thresholds differently for detecting carbon monoxide poisoning between smokers and non-smokers by the predetermined value varying depending on whether the user is a smoker.

As described above, the concentration of carboxyhemoglobin (COHb) in the blood of smokers may be higher than the concentration of carboxyhemoglobin (COHb) in the blood of non-smokers. In an embodiment, a service that shows smokers the concentration of carboxyhemoglobin (COHb) in their blood may motivate the smokers to decide to quit smoking while the concentration of carboxyhemoglobin (COHb) in their blood may be used as an indicator for assisting the smokers in quitting smoking.

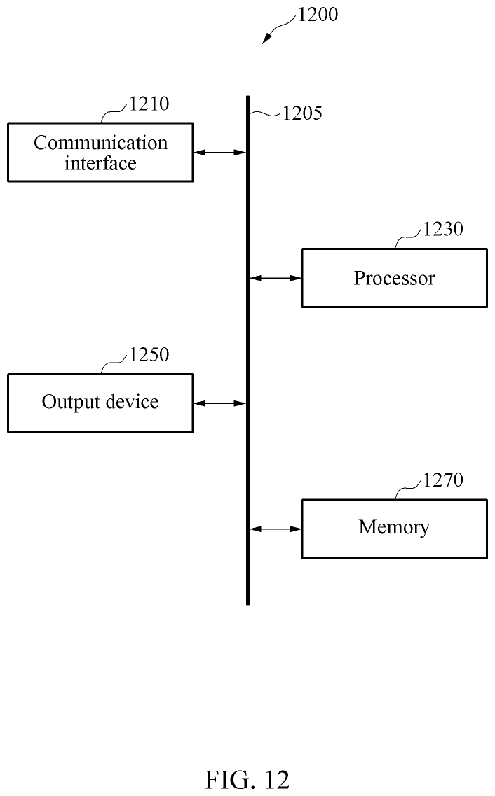

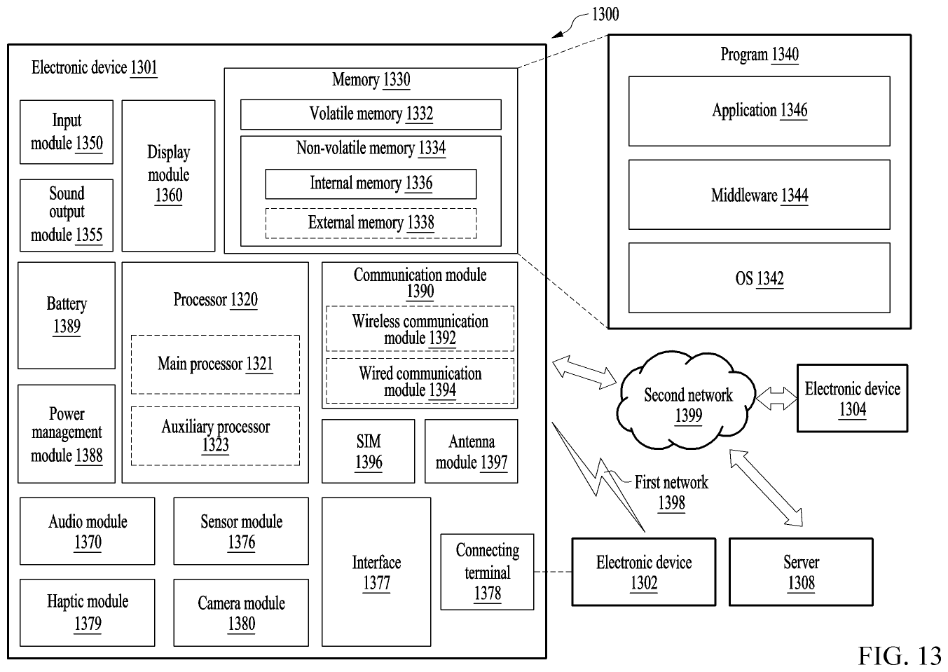

FIG. 12 is a block diagram of an embodiment of an electronic device. Referring to FIG. 12, in an embodiment, an electronic device 1200 (e.g., an electronic device 1301 of FIG. 13) may include a communication interface 1210 (e.g., a communication module 1390 of FIG. 13), a processor 1230 (e.g., a processor 1320 of FIG. 13), an output device 1250 (e.g., a sound output module 1355, a display module 1360, an audio module 1370, and/or a haptic module 1379 of FIG. 13), and a memory 1270 (e.g., a memory 1330 of FIG. 13). The communication interface 1210, the processor 1230, the output device 1250, and the memory 1270 may be connected to each other via a communication bus 1205.

The communication interface 1210 may receive pulse wave signals corresponding to lights of two or more wavelengths selected from among reflected lights at multiple wavelengths from a wearable device (e.g., the wearable device 100 of FIG. 1, the wearable device 200 of FIG. 2, and/or the wearable device 300 of FIG. 3). In an embodiment, the wearable device 300 may include a signal acquirer (e.g., the signal acquirer 340 of FIG. 3) configured to select the lights of two or more wavelengths by a difference between a second absorption rate corresponding to each of oxyhemoglobin and a reduced hemoglobin and a first absorption rate of a skin corresponding to the target from among the reflected lights, for example. The signal acquirer 340 may select the lights of two or more wavelengths, in which the second absorption rate is the same and the difference between the second absorption rate and the first absorption rate is greater than a predetermined standard, from among the reflected lights. The signal acquirer 340 may determine target wavelengths where their second absorption rates are substantially the same among the reflected lights and may calculate a first absorption rate corresponding to a target of lights of the target wavelengths. The signal acquirer 340, when the difference between the second absorption rate and the first absorption rate is greater than the predetermined standard, may select the lights of the target wavelengths. In this case, the wearable device 300 may be the wearable device 300 described above with reference to FIGS. 3 to 10, for example.

The processor 1230 may estimate the percentage of a target based on a ratio of the absorption rate of each wavelength of the pulse wave signals. In an embodiment, the processor may include an estimator (e.g., the estimator 350 of FIG. 3). The processor 1230 may calculate risk information corresponding to the target, based on the percentage of the target, for example. In an embodiment, the processor 1230 may select lights of two or more wavelengths, in which the first absorption rate of a light corresponding to the target to be detected is not changed by a second absorption rate of each of oxyhemoglobin and reduced hemoglobin, from among the reflected lights detected by the wearable device 300 and may acquire the pulse wave signals corresponding to lights of the selected wavelengths.

The output device 1250 may provide a notification to a user according to the risk information calculated by the processor 1230. The output device 1250 may include a speaker, a haptic device, and a display device, for example, but examples are not limited thereto.

The memory 1270 may store various pieces of information generated during the processing process of the processor 1230. In addition, the memory 1270 may store various pieces of data and programs. The memory 1270 may include a volatile memory or a non-volatile memory. The memory 1270 may include a high-capacity storage medium, such as a hard disk, to store the various pieces of data.

The processor 1230 may execute a program and may control the electronic device 1200. Program code to be executed by the processor 1230 may be stored in the memory 1270.

The electronic device 1200 may be a hardware-implemented electronic device having a physically structured circuit to execute operations desired by the processor 1230. In an embodiment, the desired operations may include code or instructions in a program, for example. The hardware-implemented processor 1230 may include a microprocessor, a CPU, a GPU, a processor core, a multi-core processor, a multiprocessor, an ASIC, an FPGA, and/or an NPU, for example.

FIG. 13 is a block diagram illustrating an embodiment of an electronic device 1301 in a network environment. Referring to FIG. 13, the electronic device 1301 (e.g., the electronic device 1200 of FIG. 12) in a network environment 1300 may communicate with an electronic device 1302 via a first network 1398 (e.g., a short-range wireless communication network) or at least one of an electronic device 1304 or a server 1308 via a second network 1399 (e.g., a long-range wireless communication network). In an embodiment, the electronic device 1301 may communicate with the electronic device 1304 via the server 1308. In an embodiment, the electronic device 1301 may include the processor 1320 (e.g., the processor 1230 of FIG. 12), the memory 1330 (e.g., the memory 1270 of FIG. 12), an input module 1350, a sound output module 1355, a display module 1360, an audio module 1370, and a sensor module 1376, an interface 1377, a connecting terminal 1378, a haptic module 1379, a camera module 1380, a power management module 1388, a battery 1389, a communication module 1390, a subscriber identification module (SIM) 1396, or an antenna module 1397. In an embodiment, at least one of the components (e.g., the connecting terminal 1378) may be omitted from the electronic device 1301, or one or more other components may be added in the electronic device 1301. In an embodiment, some of the components (e.g., the sensor module 1376, the camera module 1380, or the antenna module 1397) may be integrated as a single component (e.g., the display module 1360).

The processor 1320 may execute software (e.g., a program 1340) to control at least one other component (e.g., a hardware or software component) of the electronic device 1301 connected to the processor 1320 and may perform various data processing or computation, for example. In an embodiment, as at least a part of data processing or computation, the processor 1320 may store a command or data received from another component (e.g., the sensor module 1376 or the communication module 1390) in a volatile memory 1332, process the command or the data stored in the volatile memory 1332, and store resulting data in a non-volatile memory 1334. In an embodiment, the processor 1320 may include a main processor 1321 (e.g., a CPU or an application processor (AP)), or an auxiliary processor 1323 (e.g., a GPU, an NPU, an image signal processor (ISP), a sensor hub processor, or a communication processor (CP)) that is operable independently from, or in conjunction with the main processor 1321. In an embodiment, when the electronic device 1301 includes the main processor 1321 and the auxiliary processor 1323, the auxiliary processor 1323 may be adapted to consume less power than the main processor 1321 or to be predetermined to a specified function, for example. The auxiliary processor 1323 may be implemented separately from the main processor 1321 or as a part of the main processor 1321.

The auxiliary processor 1323 may control at least some of functions or states related to at least one (e.g., the display module 1360, the sensor module 1376, or the communication module 1390) of the components of the electronic device 1301, instead of the main processor 1321 while the main processor 1321 is in an inactive (e.g., sleep) state or along with the main processor 1321 while the main processor 1321 is an active state (e.g., executing an application). In an embodiment, the auxiliary processor 1323 (e.g., an ISP or a CP) may be implemented as a portion of another component (e.g., the camera module 1380 or the communication module 1390) that is functionally related to the auxiliary processor 1323. In an embodiment, the auxiliary processor 1323 (e.g., an NPU) may include a hardware structure specified for artificial intelligence (AI) model processing. An AI model may be generated by machine learning. Such learning may be performed by, e.g., the electronic device 1301 in which an AI model is executed, or performed via a separate server (e.g., the server 1308), for example. Learning algorithms may include, but are not limited to, supervised learning, unsupervised learning, semi-supervised learning, or reinforcement learning, for example. The AI model may include a plurality of artificial neural network layers. An artificial neural network may include, e.g., a deep neural network (DNN), a convolutional neural network (CNN), a recurrent neural network (RNN), a restricted Boltzmann machine (RBM), a deep belief network (DBN), a bidirectional recurrent deep neural network (BRDNN), and a deep Q-network or a combination of two or more thereof, for example, but is not limited thereto. The AI model may additionally or alternatively include a software structure other than the hardware structure.

The memory 1330 may store various data used by at least one component (e.g., the processor 1320 or the sensor module 1376) of the electronic device 1301. The various data may include software (e.g., the program 1340) and input data or output data for a command related thereto, for example. The memory 1330 may include the volatile memory 1332 or the non-volatile memory 1334.

The program 1340 may be stored as software in the memory 1330, and may include an operating system (OS) 1342, middleware 1344, or an application 1346, for example.

The input module 1350 may receive a command or data to be used by another component (e.g., the processor 1320) of the electronic device 1301, from the outside (e.g., a user) of the electronic device 1301. The input module 1350 may include a microphone, a mouse, a keyboard, a key (e.g., a button), or a digital pen (e.g., a stylus pen), for example.

The sound output module 1355 may output a sound signal to the outside of the electronic device 1301. The sound output module 1355 may include a speaker or a receiver, for example. The speaker may be used for general purposes, such as playing multimedia or playing record. The receiver may be used to receive an incoming call. In an embodiment, the receiver may be implemented separately from the speaker or as a part of the speaker.

The display module 1360 may visually provide information to the outside (e.g., a user) of the electronic device 1301. The display module 1360 may include a control circuit for controlling a display, a hologram device, or a projector and control circuitry to control a corresponding one of the display, the hologram device, and the projector, for example. In an embodiment, the display module 1360 may include a touch sensor adapted to detect a touch, or a pressure sensor adapted to measure the intensity of force incurred by the touch.

The audio module 1370 may convert a sound into an electrical signal or vice versa. In an embodiment, the audio module 1370 may obtain the sound via the input module 1350 or output the sound via the sound output module 1355 or an external electronic device (e.g., the electronic device 1302 such as a speaker or a headphone) directly or wirelessly connected to the electronic device 1301.

The sensor module 1376 may detect an operational state (e.g., power or temperature) of the electronic device 1301 or an environmental state (e.g., a state of a user) external to the electronic device 1301 and generate an electric signal or data value corresponding to the detected state. In an embodiment, the sensor module 1376 may include a gesture sensor, a gyro sensor, an atmospheric pressure sensor, a magnetic sensor, an acceleration sensor, a grip sensor, a proximity sensor, a color sensor, an IR sensor, a biometric sensor, a temperature sensor, a humidity sensor, or an illuminance sensor, for example.

The interface 1377 may support one or more specified protocols to be used for the electronic device 1301 to be coupled with the external electronic device (e.g., the electronic device 1302) directly (e.g., by wire) or wirelessly. In an embodiment, the interface 1377 may include an HDMI, a USB interface, an SD card interface, or an audio interface, for example.

The connecting terminal 1378 may include a connector via which the electronic device 1301 may be physically connected to an external electronic device (e.g., the electronic device 1302). In an embodiment, the connecting terminal 1378 may include an HDMI connector, a USB connector, an SD card connector, or an audio connector (e.g., a headphone connector), for example.

The haptic module 1379 may convert an electrical signal into a mechanical stimulus (e.g., a vibration or a movement) or an electrical stimulus which may be recognized by a user via his or her tactile sensation or kinesthetic sensation. In an embodiment, the haptic module 1379 may include a motor, a piezoelectric element, or an electric stimulator, for example.

The camera module 1380 may capture a still image and moving images. In an embodiment, the camera module 1380 may include one or more lenses, image sensors, image signal processors, or flashes.

The power management module 1388 may manage power supplied to the electronic device 1301. In an embodiment, the power management module 1388 may be implemented as at least a part of a power management integrated circuit (PMIC), for example.

The battery 1389 may supply power to at least one component of the electronic device 1301. In an embodiment, the battery 1389 may include a primary cell which is not rechargeable, a secondary cell which is rechargeable, or a fuel cell, for example.

The communication module 1390 may support establishing a direct (e.g., wired) communication channel or a wireless communication channel between the electronic device 1301 and the external electronic device (e.g., the electronic device 1302, the electronic device 1304, or the server 1308) and performing communication via the established communication channel. The communication module 1390 may include one or more communication processors that are operable independently of the processor 1320 (e.g., an AP) and that support a direct (e.g., wired) communication or a wireless communication. In an embodiment, the communication module 1390 may include a wireless communication module 1392 (e.g., a cellular communication module, a short-range wireless communication module, or a global navigation satellite system (GNSS) communication module) or a wired communication module 1394 (e.g., a local area network (LAN) communication module, or a power line communication (PLC) module). A corresponding one of these communication modules may communicate with the external electronic device 1304 via the first network 1398 (e.g., a short-range communication network, such as Bluetooth™, wireless-fidelity (Wi-Fi) direct, or IR data association (IrDA)) or the second network 1399 (e.g., a long-range communication network, such as a legacy cellular network, a 5G network, a next-generation communication network, the Internet, or a computer network (e.g., a LAN or a wide area network (WAN)). These various types of communication modules may be implemented as a single component (e.g., a single chip) or may be implemented as multiple components (e.g., multiple chips) separate from each other. The wireless communication module 1392 may identify and authenticate the electronic device 1301 in a communication network, such as the first network 1398 or the second network 1399, using subscriber information (e.g., international mobile subscriber identity (IMSI)) stored in the SIM 1396.

The wireless communication module 1392 may support a 5G network after a 4G network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 1392 may support a high-frequency band (e.g., a mmWave band) to achieve, e.g., a relatively high data transmission rate. The wireless communication module 1392 may support various technologies for securing performance on a high-frequency band, such as, e.g., beamforming, massive multiple-input and multiple-output (massive MIMO), full dimensional MIMO (FD-MIMO), an array antenna, analog beamforming, or a large-scale antenna. The wireless communication module 1392 may support various requirements specified in the electronic device 1301, an external electronic device (e.g., the electronic device 1304), or a network system (e.g., the second network 1399). In an embodiment, the wireless communication module 1392 may support a peak data rate (e.g., 20 gigabits per second (Gbps) or more) for implementing eMBB, loss coverage (e.g., 164 dB or less) for implementing mMTC, or U-plane latency (e.g., 0.5 millisecond (ms) or less for each of downlink (DL) and uplink (UL), or a round trip of 1 ms or less) for implementing URLLC.

The antenna module 1397 may transmit or receive a signal or power to or from the outside (e.g., the external electronic device) of the electronic device 1301. In an embodiment, the antenna module 1397 may include an antenna including a radiating element including a conductive material or a conductive pattern formed in or on a substrate (e.g., a PCB). In an embodiment, the antenna module 1397 may include a plurality of antennas (e.g., array antennas). In such a case, at least one antenna appropriate for a communication scheme used in a communication network, such as the first network 1398 or the second network 1399, may be selected by the communication module 1390 from the plurality of antennas, for example. The signal or the power may be transmitted or received between the communication module 1390 and the external electronic device via the at least one selected antenna. In an embodiment, another component (e.g., a radio frequency integrated circuit (RFIC)) other than the radiating element may be additionally formed as a part of the antenna module 1397.

According to various embodiments, the antenna module 1397 may form a mmWave antenna module. In an embodiment, the mmWave antenna module may include a PCB, an RFIC on a first surface (e.g., the bottom surface) of the PCB, or next (adjacent) to the first surface of the PCB and capable of supporting a designated high-frequency band (e.g., a mmWave band), and a plurality of antennas (e.g., array antennas) arranged on a second surface (e.g., the top or a side surface) of the PCB, or next (adjacent) to the second surface of the PCB and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

In an embodiment, commands or data may be transmitted or received between the electronic device 1301 and the external electronic device 1304 via the server 1308 coupled with the second network 1399. Each of the external electronic devices 1302 or 1304 may be a device of the same type as or a different type from the electronic device 1301. In an embodiment, all or some of operations to be executed by the electronic device 1301 may be executed at one or more of the external electronic devices 1302 and 1304, and the server 1308. In an embodiment, when the electronic device 1301 needs to perform a function or a service automatically, or in response to a request from a user or another device, the electronic device 1301, instead of, or in addition to, executing the function or the service, may request one or more external electronic devices to perform at least part of the function or the service, for example. The one or more external electronic devices receiving the request may perform the at least part of the function or the service requested, or an additional function or an additional service related to the request and may transfer an outcome of the performing to the electronic device 1301.

The electronic device 1301 may provide the outcome, with or without further processing of the outcome, as at least part of a reply to the request. To that end, a cloud computing, distributed computing, mobile edge computing (MEC), or client-server computing technology may be used, for example. The electronic device 1301 may provide ultra-low-latency services using, e.g., distributed computing or MEC. In an embodiment, the external electronic device 1304 may include an IoT device. The server 1308 may be an intelligent server using machine learning and/or a neural network. In an embodiment, the external electronic device 1304 or the server 1308 may be included in the second network 1399. The electronic device 1301 may be applied to intelligent services (e.g., smart home, smart city, smart car, or healthcare) based on 5G communication technology or IoT-related technology.

In an embodiment, a wearable device 100, 200, or 300 includes a light emitter 310, 410, 420, 430, 440, or 510 including a light source 315 that emits lights of multiple wavelengths including any one of a first wavelength band corresponding to a first bandwidth (420 nm-460 nm) and a second wavelength band corresponding to a second bandwidth (500 nm-540 nm) and a third wavelength band corresponding to a third bandwidth (785 nm-825 nm), a light receiver 320, 460, or 530 configured to detect at least some of reflected lights that are reflected from a user's skin by the lights of multiple wavelengths, a signal acquirer 340 configured to select lights of two or more wavelengths, in which an absorption rate of oxyhemoglobin is the same as an absorption rate of a reduced hemoglobin, from among the reflected lights and acquire pulse wave signals corresponding to the selected lights of wavelengths, and an estimator 350 configured to estimate a percentage (%) of a target to be detected, based on a ratio of an absorption rate of each wavelength of the pulse wave signals.

In an embodiment, the signal acquirer 340 may select any one of the third wavelength band, in which a second absorption rate of the oxyhemoglobin and the reduced hemoglobin is different from a first absorption rate of a carboxyhemoglobin, and the first wavelength band or the second wavelength band, in which the second absorption rate is the same as the first absorption rate, from among the reflected lights.

In an embodiment, among the reflected lights, the signal acquirer 340 may determine a light of the first wavelength band or a light of the second wavelength band where the second absorption rate is substantially the same as the first absorption rate to be a light of a first wavelength, may determine a light of the third wavelength band where the second absorption rate is not the same as the first absorption rate to be a light of a second wavelength, may calculate the first absorption rate corresponding to the target in the light of the first wavelength and the light of the second wavelength, and, when a difference between the second absorption rate and the first absorption rate is greater than a predetermined standard, may select the light of the first wavelength and the light of the second wavelength.

In an embodiment, the first wavelength may be about 520 nm, and the second wavelength may be about 805 nm.

In an embodiment, the light emitters 310, 410, 420, 430, 440, and 510 may include a plurality of the light emitters 310, 410, 420, 430, 440, and 510, and the wearable device 100, 200, or 300 may operate the light emitters 310, 410, 420, 430, 440, and 510 corresponding to different wavelengths at regular time intervals and may acquire the pulse wave signals by reflected lights detected by the light receivers 320, 460, and 530, based on the operation of the light emitters 310, 410, 420, 430, 440, and 510.

In an embodiment, the wearable device 100, 200, or 300 may include an analog circuit that performs signal processing including signal amplification and filtering for the detected reflected lights and an analog circuit & ADC converter that converts the signal-processed reflected lights into a digital signal.

In an embodiment, the light source 315 may include any one of a single LED light source 315 that emits a light of a single wavelength band, multiple LED light sources 315 that emit a light of a single wavelength band, and a multispectral light source 315 that emits multiple wavelengths in a spectral sensor.

In an embodiment, the target may include carboxyhemoglobin (COHb).

In an embodiment, the estimator 350 may estimate a percentage of the target of the total hemoglobin (Hb) including oxyhemoglobin and reduced hemoglobin, based on a ratio of an absorption rate of each wavelength of the pulse wave signals.

In an embodiment, the estimator 350 may calculate relative absorption rates by a volume change by each wavelength, based on the pulse wave signals, and may estimate the percentage of the target by applying the relative absorption rates to pre-acquired information corresponding to the target.

In an embodiment, the information corresponding to the target may include a difference between a pre-calculated ratio and a correlation coefficient of the concentration of the target.

In an embodiment, the wearable device 100, 200, or 300 may define risk information, including mild, moderate, severe, and fatal, of the user depending on the size of an estimated percentage of the target.

In an embodiment, the wearable device 100, 200, or 300 may determine whether the user is sleeping, based on the user's biosignal, when the user is determined to be sleeping, may determine whether the percentage of the target increases by a set threshold or more during a predetermined time interval, and, when the percentage of the target is determined to increase by the threshold or more, may notify the user being in a risk state by the target.

In an embodiment, the wearable device 100, 200, or 300 may include the notifier 360 that provides the risk information corresponding to the target as a notification, based on the percentage of the target.

In an embodiment, the notifier 360 may notify the user of the risk information corresponding to the target by at least one notification method among screen flickering, screen flashing, haptic notification by touch, and alarm sound or notify a preset contact person of the risk information corresponding to the target through communication connection or message transmission to a designated contact.

In an embodiment, the wearable device 100, 200, or 300 may include the communication interface 1210 configured to receive pulse wave signals corresponding to lights of two or more wavelengths selected from among reflected lights by lights of multiple wavelengths from a wearable device, the processor 1230 configured to estimate a percentage of a target to be detected, based on a ratio of an absorption rate of each wavelength of the pulse wave signals, and calculate risk information corresponding to the target, based on the percentage of the target, and the output device 1250 configured to provide a notification to a user according to the risk information.

In an embodiment, the wearable device 100, 200, or 300 may include the signal acquirer 340 configured to select the lights of two or more wavelengths by a difference between a second absorption rate corresponding to each of oxyhemoglobin and reduced hemoglobin and a first absorption rate of the skin corresponding to the target from among the reflected lights.

In an embodiment, the signal acquirer 340 may select the lights of two or more wavelengths, in which the second absorption rate is the same and the difference between the second absorption rate and the first absorption rate is greater than a predetermined standard, from among the reflected lights.

In an embodiment, the signal acquirer 340 may determine target wavelengths, in which the second absorption rate is substantially the same, among the reflected lights, may calculate the first absorption rate corresponding to the target of lights of the target wavelengths, and, when the difference between the second absorption rate and the first absorption rate is greater than the predetermined standard, may select the lights of the target wavelengths.

In an embodiment, an operating method of the wearable device 100, 200, or 300 may include an operation (operation 1010 of FIG. 10) of irradiating the user' skin with lights of multiple wavelengths from the light source 315, an operation (operation 1020 of FIG. 10) of detecting reflected lights reflected from the skin by the lights of multiple wavelengths, an operation (operation 1030 of FIG. 10) of selecting lights of two or more wavelengths, in which an absorption rate of oxyhemoglobin is the same as an absorption rate of a reduced hemoglobin, from among the reflected lights, an operation (operation 1040 of FIG. 10) of acquiring pulse wave signals corresponding to the selected lights of wavelengths, an operation (operation 1050 of FIG. 10) of estimating a percentage of a target to be detected, based on a ratio of an absorption rate of each wavelength of the pulse wave signals, and an operation (operation 1060 of FIG. 10) of notifying risk information corresponding to the target, based on the percentage of the target.

The wearable device or the electronic device in the embodiments disclosed herein may be one of various types of devices.

The electronic device may include a portable communication device (e.g., a smartphone), a computer device, a portable multimedia device, a portable medical device, a camera, a wearable device, or a home appliance device, for example. In an embodiment of the disclosure, the electronic device is not limited to those described above. The wearable device may be necklace-type, watch-type, bracelet-type, ring-type and/or glasses-type, for example, but examples are not limited thereto.

It should be appreciated that an embodiment of the disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, “A or B”, “at least one of A and B”, “at least one of A or B”, “A, B or C”, “at least one of A, B and C”, and “A, B, or C,” each of which may include any one of the items listed together in the corresponding one of the phrases, or all possible combinations thereof. Terms such as “first”, “second”, or “first” or “second” may simply be used to distinguish the component from other components in question and may refer to components in other features (e.g., importance or order) is not limited. It is to be understood that when an element (e.g., a first element) is referred to, with or without the term “operatively” or “communicatively”, as “coupled with,” “coupled to,” “connected with,” or “connected to” another element (e.g., a second element), it means that the element may be coupled with a remaining (the other) element directly (e.g., by wire), wirelessly, or via a third element.

As used in connection with embodiments of the disclosure, the term “module” may include a unit implemented in hardware, software, or firmware, and may interchangeably be used with other terms such as “logic”, “logic block”, “part”, or “circuitry”. A module may be a single integral component, or a minimum unit or part thereof, adapted to perform one or more functions. In an embodiment, the module may be implemented in a form of an ASIC, for example. Further, the terms such as an estimator, a communication interface, a processor, an output device may be hardware components such as circuitry.

An embodiment as set forth herein may be implemented as software (e.g., the program 1340) including one or more instructions that are stored in a storage medium (e.g., internal memory 1336 or external memory 1338) that is readable by a machine (e.g., the electronic device 1301 of FIG. 13). In an embodiment, a processor (e.g., the processor 1320) of the machine (e.g., the electronic device 1301) may invoke at least one of the one or more instructions stored in the storage medium, and execute it, for example. This allows the machine to be operated to perform at least one function according to the at least one instruction invoked. The one or more instructions may include code generated by a compiler or code executable by an interpreter. The machine-readable storage medium may be provided in the form of a non-transitory storage medium. Here, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

A method in an embodiment of the disclosure may be included and provided in a computer program product. The computer program product may be traded as a product between a seller and a buyer. The computer program product may be distributed in the form of a machine-readable storage medium (e.g., a compact disc read-only memory (CD-ROM)), or be distributed (e.g., downloaded or uploaded) online via an application store (e.g., PlayStore™), or between two user devices (e.g., smartphones) directly. When distributed online, at least part of the computer program product may be temporarily generated or at least temporarily stored in the machine-readable storage medium, such as a memory of the manufacturer's server, a server of the application store, or a relay server.

In an embodiment, each component (e.g., a module or a program) of the above-described components may include a single entity or multiple entities, and some of the multiple entities may be separately disposed in different components. In an embodiment, one or more of the above-described components may be omitted, or one or more other components may be added. In an alternative embodiment or additionally, a plurality of components (e.g., modules or programs) may be integrated into a single component. In such a case, in embodiments, the integrated component may still perform one or more functions of each of the plurality of components in the same or similar manner as they are performed by a corresponding one of the plurality of components before the integration. In an embodiment, operations performed by the module, the program, or another component may be carried out sequentially, in parallel, repeatedly, or heuristically, or one or more of the operations may be executed in a different order or omitted, or one or more other operations may be added.

1. A wearable device which estimates a percentage of a target to be detected in a user having a skin, the wearable device comprising:
a light emitter comprising:
a light source which emits lights of multiple wavelengths, the lights of the multiple wavelengths comprising:
a first wavelength band corresponding to a first bandwidth equal to or greater than 420 nanometers and equal to or less than 460 nanometers;
a second wavelength band corresponding to a second bandwidth equal to or greater than 500 nanometers and equal to or less than 540 nanometers; and
a third wavelength band corresponding to a third bandwidth equal to or greater than 785 nanometers and equal to or less than 825 nanometers;
a light receiver configured to detect at least some of reflected lights which are reflected from the skin of the user by the lights of the multiple wavelengths;
a signal acquirer configured to:
select lights of two or more wavelengths, in which an absorption rate of oxyhemoglobin is identical to an absorption rate of a reduced hemoglobin, from among the reflected lights; and
acquire pulse wave signals corresponding to the selected lights of wavelengths; and
an estimator configured to estimate the percentage of the target to be detected, based on a ratio of an absorption rate of each wavelength of the pulse wave signals.

2. The wearable device of claim 1, wherein
the signal acquirer is configured to:
select any one of the third wavelength band, in which a second absorption rate of the oxyhemoglobin and the reduced hemoglobin is different from a first absorption rate of a carboxyhemoglobin; and
the first wavelength band or the second wavelength band, in which the second absorption rate is identical to the first absorption rate, from among the reflected lights.

3. The wearable device of claim 2, wherein
the signal acquirer is configured to:
determine a light of the first wavelength band or a light of the second wavelength band, in which the second absorption rate is substantially identical to the first absorption rate, as a light of a first wavelength among the reflected lights;
determine a light of the third wavelength band, in which the second absorption rate is not identical to the first absorption rate, as a light of a second wavelength;
calculate the first absorption rate corresponding to the target in the light of the first wavelength and the light of the second wavelength; and,
when a difference between the second absorption rate and the first absorption rate is greater than a predetermined standard, select the light of the first wavelength and the light of the second wavelength.

4. The wearable device of claim 3, wherein
the first wavelength is 520 nanometers, and
the second wavelength is 805 nanometers.

5. The wearable device of claim 1, wherein
the light emitter comprises:
multiple emitters, and
the wearable device is configured to:
operate the multiple emitters corresponding to different wavelengths at a set time interval, and
acquire the pulse wave signals by the at least some of the reflected lights detected by the light receiver, based on an operation of the multiple emitters.

6. The wearable device of claim 1, wherein
the wearable device further comprises:
an analog circuit configured to perform signal processing comprising signal amplification and filtering on the at least some of the reflected lights detected by the light receiver; and
an analog-digital converter configured to convert the signal-processed reflected lights into digital signals.

7. The wearable device of claim 1, wherein
the light source comprises any one of:
a single light-emitting diode light source configured to generate a light of a single wavelength band;
multiple light-emitting diode light sources configured to generate a light of a single wavelength band; and
a multispectral light source configured to generate multiple wavelengths in a spectral sensor.

8. The wearable device of claim 1, wherein
the target comprises:
a carboxyhemoglobin (COHb), and
the estimator is configured to:
estimate a percentage of the target of a total hemoglobin (Hb) comprising the oxyhemoglobin and the reduced hemoglobin, based on the ratio of the absorption rate of the each wavelength of the pulse wave signals.

9. The wearable device of claim 1, wherein
the estimator is configured to:
calculate a relative absorption rate by a volume change of each wavelength, based on the pulse wave signals; and
estimate the percentage of the target by applying the relative absorption rate to pre-acquired information corresponding to the target, and
the information corresponding to the target comprises:
a difference between a pre-calculated ratio and a correlation coefficient of a concentration of the target.

10. The wearable device of claim 1, wherein
the wearable device is configured to:
define risk information, comprising mild, moderate, severe, and fatal, of the user depending on a size of an estimated percentage of the target.

11. The wearable device of claim 1, wherein
the wearable device is configured to:
determine whether the user is sleeping based on biometric information of the user, determine whether the percentage of the target increases by a threshold or more when the user is determined to be sleeping, and
notify that the user is in a risk state by the target when the percentage of the target is determined to increase by the threshold or more.

12. The wearable device of claim 1, wherein
the wearable device further comprises:
a notifier configured to provide risk information corresponding to the target through a notification, based on the percentage of the target, and
the notifier is configured to:
notify the user of the risk information corresponding to the target by at least one of screen flickering, screen flashing, haptic notification by touch, and alarm sound; or
notify a preset contact person of the risk information corresponding to the target through communication connection or message transmission to a designated contact.

13. An electronic device which estimates a percentage of a target to be detected in a user having a skin, the wearable device comprising:
a communication interface configured to receive pulse wave signals corresponding to lights of two or more wavelengths selected from among reflected lights by lights of multiple wavelengths from a wearable device;
a processor configured to:
estimate the percentage of the target to be detected, based on a ratio of an absorption rate of each wavelength of the pulse wave signals; and
calculate risk information corresponding to the target, based on the percentage of the target; and
an output device configured to provide a notification to the user according to the risk information.

14. The electronic device of claim 13, wherein
the wearable device comprises:
a signal acquirer configured to select the lights of two or more wavelengths by a difference between a second absorption rate corresponding to each of oxyhemoglobin and a reduced hemoglobin and a first absorption rate of the skin corresponding to the target from among the reflected lights, and
the signal acquirer is configured to:
select the lights of two or more wavelengths, in which the second absorption rate is identical and the difference between the second absorption rate and the first absorption rate is greater than a predetermined standard, from among the reflected lights, or
the signal acquirer is configured to:
determine target wavelengths, in which the second absorption rate is substantially identical, among the reflected lights, calculate the first absorption rate corresponding to the target of lights of the target wavelengths; and
when the difference between the second absorption rate and the first absorption rate is greater than the predetermined standard, select the lights of the target wavelengths.

15. An operating method of a wearable device which estimates a percentage of a target to be detected in a user having a skin, the method comprising:
irradiating the skin of the user with lights of multiple wavelengths from a light source;
detecting reflected lights reflected from the skin by the lights of multiple wavelengths;
selecting lights of two or more wavelengths, in which an absorption rate of oxyhemoglobin is identical to an absorption rate of a reduced hemoglobin, from among the reflected lights;
acquiring pulse wave signals corresponding to the selected lights of wavelengths;
estimating the percentage of the target to be detected, based on a ratio of an absorption rate of each wavelength of the pulse wave signals; and
notifying risk information corresponding to the target, based on the percentage of the target.
